SEQUENCE LISTING (6 sequences)

SEQ ID NO: 1 - protein, 154 aa, Artificial Sequence
  Pti5-like sequence
MVPTSQSELPLNENDSQDMVIYQVLNEANALNNSFLPQRNQQISKNSLEPTRNIGKKYRGVRRRPWGKYAAEIRDSARHGARVWLGTFETAEEAALAYDRAAFRMRGAKALLNFPAEVVAASPSGSSASEIRNSSSSSSSWLSSFTTAEEASSS

SEQ ID NO: 2 - protein, 86 aa, Artificial Sequence
  SAR8.2A-like sequence
MVSKNNIFVCLSLVILLIMSSQIVAREMNSEAPAPLTQAMNGNNTSEAKGVGHHLLKGLGKIFRAGKVIYCNYCKTCHGLCDYCCV

SEQ ID NO: 3 - protein, 161 aa, Artificial Sequence
  Pti5 protein sequence
MVPTPQSDLPLNENDSQEMVLYEVLNEANALNIPYLPQRNQLLPRNNILRPLQCIGKKYRGVRRRPWGKYAAEIRDSARHGARIWLGTFETAEEAALAYDRAAFRMRGAKALLNFPSEIVNASVSVDKLSLCSNSYTTNNNSDSSLNEVSSGTNDVFESRC

SEQ ID NO: 4 - DNA, 486 bp, Artificial Sequence
  Sequence coding for Pti5 protein of Seq_3
atggttccaactccacagtctgatcttccactcaacgagaacgattctcaagagatggttctttacgaggttttgaacgaggctaacgctcttaacattccatacctcccacaaagaaaccagcttctccctaggaacaacattcttaggccacttcagtgcattggaaagaagtacaggggagttagaagaaggccttggggaaagtacgctgctgagattagagattctgctagacacggtgctagaatttggcttggaactttcgaaactgctgaagaagctgctcttgcttacgatagagctgctttcagaatgagaggtgctaaggctcttttgaacttcccatccgagattgtgaacgcttctgtgtctgtggataagctttctctttgctccaactcttacaccaccaacaacaactctgattctagccttaacgaggtttcctctggaactaacgatgttttcgagtccaggtgctaa

SEQ ID NO: 5 - protein, 86 aa, Artificial Sequence
  SAR8.2 protein sequence
MVSKSSIFICLSLIILLIMSTQIVAREMTSEASASLTQAMNGNNISETKKVGRHLVKGLGKIFKAGKVIYCKTCKTCHGRCDYCCA

SEQ ID NO: 6 - DNA, 261 bp, Artificial Sequence
  Sequence coding for the SAR8.2A protein of Seq_5
atggttagtaagtctagtatctttatctgccttagcctgattatcctcctgattatgagcactcagatcgtggctagggaaatgactagtgaggctagcgctagtctgactcaggctatgaacggtaacaatattagcgagactaagaaggtgggccgtcaccttgttaagggactcggtaagatctttaaggccggtaaggtgatctactgtaagacttgtaagacctgtcacggtaggtgcgattactgctgcgcttaa

(19) United States
(12) Patent Application Publication
(10) Pub. No.: US 2024/0287538 A1
(43) Pub. Date: Aug. 29, 2024

(54) YIELD IMPROVEMENT BY GENE COMBINATIONS

(71) Applicant: BASF SE, Ludwigshafen (DE)

(72) Inventors: Brody John DeYoung, Research Triangle Park, NC (US); Renata Bocci Zanon, Santo Antonio De Posse (BR); Yunxing Cory Cui, Research Triangle Park, NC (US); Holger Schultheiss, Einbeck (DE)

(21) Appl. No.: 18/569,926

(22) PCT Filed: Jun. 9, 2022
(86) PCT No.: PCT/EP2022/065687

§ 371 Date: Dec. 13, 2023

Related U.S. Application Data
(60) Provisional application No. 63/210,291, filed on Jun. 14, 2021.

(51) Int. Cl. C12N 15/82; C07K 14/415

(52) U.S. Cl. CPC C12N 15/8282; C07K 14/415

(57) ABSTRACT

The present invention relates to plant breeding and farming. In particular the invention relates to materials and methods for improving plant yield. Preferably such improvement is visible under fungal pathogen stress.

### FIELD OF THE INVENTION

The present invention relates to plant breeding and farming. In particular, the invention relates to materials and methods for improving plant yield. Preferably, such improvement is visible under fungal pathogen stress.

### BACKGROUND OF THE INVENTION

Plant pathogenic organisms, in particular fungi, have resulted in severe reductions in crop yield in the past, in worst cases leading to famine. Monocultures, in particular, are highly susceptible to an epidemic-like spreading of diseases. To date, the pathogenic organisms have been controlled mainly by using pesticides. Currently, the possibility of directly modifying the genetic disposition of a plant or pathogen is also open to man. Alternatively, naturally occurring fungicides produced by the plants after fungal infection can be synthesized and applied to the plants.

Yield is affected by various factors, for example the number and size of the plant organs, plant architecture (for example, the number of branches), number of filled seed or grains, plant vigor, growth rate, root development, utilization of water and nutrients and especially abiotitic and biotic stress tolerance.

In the past efforts have been made to create plants resistant against biotic stresses such as fungal pathogens. The term “resistance” as used herein refers to an absence or reduction of one or more disease symptoms in a plant caused by a plant pathogen. Resistance generally describes the ability of a plant to prevent, or at least curtail the infestation and colonization by a harmful pathogen. Different mechanisms can be discerned in the naturally occurring resistance, with which the plants fend off colonization by phytopathogenic organisms (Schopfer and Brennicke (1999) Pflanzenphysiologie, Springer Verlag, Berlin-Heidelberg, Germany). In nature, however, resistance is often overcome because of the rapid evolutionary development of new virulent races of the pathogens, including fungi (Neu et al. (2003) American Cytopathol. Society, MPMI 16 No. 7: 626-633).

Fungi are distributed worldwide. Approximately 100 000 different fungal species are known to date. Thereof, rusts are of great importance. They can have a complicated development cycle with up to five different spore stages (spermatium, aecidiospore, uredospore, teleutospore and basidiospore). Specific infection structures are developed for penetration of the plant. Biotrophic phytopathogenic fungi depend on the metabolism of living plant cells for their nutrition. Examples of biotrophic fungi include many rust fungi, powdery mildew fungi or oomycete pathogens like the genus Phytophthora or Peronospora. Necrotrophic phytopathogenic fungi depend for their nutrition on dead cells of the plants, e.g. species from the genus Fusarium, Rhizoctonia or Mycosphaerella. Soybean rust occupies an intermediate position. It penetrates the epidermis directly, whereupon the penetrated cell becomes necrotic. However, after penetration, the fungus changes over to an obligate-biotrophic lifestyle. The subgroup of the biotrophic fungal pathogens which follows essentially such an infection strategy are heminecrotrophic.

The soybean rust, Phakopsora pachyrhizi, directly penetrates the plant epidermis. After growing through the epidermal cell, the fungus reaches the intercellular space of the mesophyll, where the fungus starts to spread through the leaf. To acquire nutrients, the fungus penetrates mesophyll cells and develops haustoria inside the mesophyll cells. It is a particularly troubling feature of Phakopsora pachyrhizi that this pathogen exhibits an immense variability, thereby overcoming novel plant resistance mechanisms and novel fungicide activities within a few years and sometimes already within one Brazilian growing season.

Despite the scientific importance of resistance, resistance is only of economic value if it leads to increased crop yields or crop quality (in comparison to susceptible varieties), when the disease is present.

With progress in increasing resistance in crop plants it became clear that improvements in fungal resistance are not correlated to improvements in yield, in particular under natural in-field growth conditions instead of sheltered greenhouse environments. Even genes which reliably lead to a strong fungal resistance may not increase, or may even decrease, yield. Contrary to common sense and speculations and assertions in literature, the traits of fungal resistance and yield are in the best case independent from each other, but often even counteracting (for a review on this topic see Ning et al. Balancing Immunity and Yield in Crop Plants Trends in Plant Science 22(12), 1069-1079). Farmers, however, are mainly interested in yield. The degree by which plants are affected by fungal infections is of no concern unless yield is also affected.

In the past, multiple genes were identified that increased resistance of soybean to soybean rust, examples of such publications are WO2014118018, WO2013001435, WO2014076614, WO2014024079 and WO2012023099.

However, as shown in the examples it is not possible to predict, with any significant confidence, the development of yield by expression of a gene responsible for resistance against a fungal pathogen. Thus, the trait of yield improvement is independent from and cannot be predicted by the trait of fungal resistance. Furthermore, as also shown herein, combinations of genes individually involved in yield increase generally do not lead to a super-additive yield improvement, and frequently even result in a yield increase less than the theoretical additive yield increase effect predicted from the individual genes. In fact, co-expression of genes individually involved in yield increase and fungal resistance can even lead to a yield decrease.

It was thus the object of the invention to provide materials and methods to improve plant yield, particularly for crops and preferably providing yield increases despite potential fungal pathogen stress. In particular, it was a preferred object of the invention to provide materials and methods which lead to plant material of heritably improved yield even under conditions of infection by a fungal pathogen, preferably a rust fungus and most preferably a rust fungus of genus Phakopsora, but also under conditions without significant infection pressure.

### SUMMARY OF THE INVENTION

The inventors have found that certain genes provide yield improvements in plants, in particular in crops. Notably the simultaneous presence of Pti5 and SAR8.2 proteins in the cells of a plant, preferably a crop plant, more preferably a crop plant outside the taxonomic sub-family Solanoidae, is shown herein to surprisingly improve seed yield under natural fungal pathogen stress conditions.

Thus, the following teachings of the invention are encompassed by the disclosure:

The invention provides a method for improving the yield produced by a plant relative to a control plant, comprising
i) providing a plant comprising a Pti5 and a SAR8.2 gene and/or a Pti5-SAR8.2 fusion gene, wherein preferably the Pti5 and/or SAR8.2 genes are provided in a respective heterologous expression cassette, and
ii) cultivating the plant.

The invention also provides a plant cell, plant part or whole plant comprising a Pti5 and a SAR8.2 gene and/or a Pti5-SAR8.2 fusion gene, wherein the plant preferably comprises a heterologous Pti5 expression cassette and/or a heterologous SAR8.2 expression cassette.

Also provided according to the invention is a method for producing a hybrid plant having improved yield relative to a control plant, comprising
i) providing
i-a) a first plant material comprising a Pti5 and a SAR8.2 gene and/or a Pti5-SAR8.2 fusion gene, preferably comprising a heterologous Pti5 expression cassette and a heterologous SAR8.2 expression cassette, and a second plant material not comprising both a Pti5 and a SAR8.2 gene or a Pti5-SAR8.2 fusion gene, or
i-b) a first plant material comprising a Pti5 gene, preferably comprising a heterologous Pti5 expression cassette, and a second plant material comprising a SAR8.2 gene, preferably comprising a heterologous SAR8.2 expression cassette,
ii) producing an F1 generation from a cross of the first and second plant material, and
iii) selecting one or more members of the F1 generation capable of expression of Pti5 and SAR8.2.

The invention furthermore provides the use of a combination of at least a Pti5 gene and a SAR8.2 gene, a Pti5-SAR8.2 fusion gene or a plant, plant part or plant cell according to the invention for improving yield of a plant, preferably under natural field conditions, more preferably under pathogen pressure, more preferably wherein at least in one plant growth stage the average diseased leaf area is 2-100%, more preferably 5-50%, more preferably 10-50%, wherein yield is one or more of
biomass per area,
grain mass per area,
seed mass per area,
   preferably seed mass per area.

And the invention provides a method of synergistic yield improvement comprising expressing, in a plant cell, plant part or plant at least a Pti5 protein and a SAR8.2 protein.

### BRIEF DESCRIPTION OF FIGURES

FIG. 1 shows the relative disease resistance provided by the expression of Pti5, SAR8.2 and the combination of SAR8.2 and Pti5 under 2 different treatments

To compare the disease progression over the whole season in plants expressing the single genes or the combination of SAR8.2 and Pti5 in comparison to the wild type, the relative disease resistance (average relative disease resistance=(AUDPC(control)/AUDPC(event))−1)*100% averaged over locations) was calculated.

It is clearly visible that both single genes provide increased resistance in both treatments (untreated: no fungicide treatment, A treatment: one fungicide treatment at the onset of ASR disease (˜35-40 days after planting)). By comparing the relative disease resistance of the single gene expressing variants with the variants expressing both genes it becomes clear that the disease resistance does not combine in an additive (or more than additive) way.

FIG. 2 shows the Colby formula that is commonly used to predict the total trait efficacy for 2 factors that act additively on the same trait.

FIG. 3a shows the relative yield increase [%] of soybean expressing the single genes Pti5 or SAR8.2 or the combination of both genes (SAR8.2+Pti5) in comparison to non-transgenic wildtype soybean (average yield increase=(yield(control)/yield(event)−1)*100%), with and without fungicide treatment (untreated: no fungicide treatment, A treatment: one fungicide treatment at the onset of ASR disease (˜35-40 days after planting)). The dotted bar shows the predicted relative yield increase based on the Colby Formula (see FIG. 2) when using the yield increases mediated by both single genes. As the dotted bar is lower than the diagonally striped bar (showing the real measured yield increase mediated by the combination of Pti5 and SAR8.2 (stack)), the result can be considered more than additive. The graph shows the results measured at location 1.

It is clearly visible that the yield increase mediated by the combination of Pti5 and SAR8.2 is larger than the additive yield increase that is predicted by the Colby formula based on the performance of the single genes. So, the combination of SAR8.2 and Pti5 leads to more than additive yields.

FIG. 3b shows the relative yield increase [%] of soybean expressing the single genes Pti5 or SAR8.2 or the combination of both genes (SAR8.2+Pti5) in comparison to non-transgenic wildtype soybean, with and without fungicide treatment (untreated: no fungicide treatment, A treatment: one fungicide treatment at the onset of ASR disease (˜35-40 days after planting)). The dotted bar shows the predicted relative yield increase based on the Colby Formula (see FIG. 2) when using the yield increases mediated by both single genes. As the dotted bar is lower than the diagonally striped bar showing the real measured yield increase mediated by the combination of Pti5 and SAR8.2 (stack), the result can be considered more than additive. The graph shows the results measured at location 2.

It is clearly visible that the yield increase mediated by the combination of Pti5 and SAR8.2 is larger than the additive yield increase that is predicted by the Colby formula based on the performance of the single genes. So, the combination of SAR8.2 and Pti5 leads to more than additive yields.

FIG. 4a shows the relative yield increase [%] of soybean expressing the single genes Pti5 or ADR1 or the combination of both genes (ADR1+Pti5) in comparison to non-transgenic wildtype soybean, with and without fungicide treatment (untreated: no fungicide treatment, A treatment: one fungicide treatment at the onset of ASR disease (˜35-40 days after planting)). The dotted bar shows the predicted relative yield increase based on the Colby Formula (see FIG. 2) when using the yield increases mediated by both single genes. If the dotted bar would be lower than the diagonally striped bar showing the real measured yield increase mediated by the combination of Pti5 and ADR1 (stack), the result could be considered more than additive. The graph shows the results measured at location 1.

It is clearly visible that the yield increase mediated by the combination of Pti5 and ADR1 is much lower than the additive yield increase that is predicted by the Colby formula based on the performance of the single genes. So, the combination of ADR1 and Pti5 leads to less than additive yields.

FIG. 4b shows the relative yield increase [%] of soybean expressing the single genes Pti5 or ADR1 or the combination of both genes (ADR1+Pti5) in comparison to non-transgenic wildtype soybean, with and without fungicide treatment (untreated: no fungicide treatment, A treatment: one fungicide treatment at the onset of ASR disease (˜35-40 days after planting)). The dotted bar shows the predicted relative yield increase based on the Colby Formula (see FIG. 2) when using the yield increases mediated by both single genes. If the dotted bar is lower than the diagonally striped bar showing the real measured yield increase mediated by the combination of Pti5 and ADR1 (stack), the result can be considered more than additive. The graph shows the results measured at location 2.

It is clearly visible that the yield increase mediated by the combination of Pti5 and ADR1 is much lower than the additive yield increase that is predicted by the Colby formula based on the performance of the single genes. So, the combination of ADR1 and Pti5 leads to less than additive yields.

FIG. 5a shows the relative yield increase [%] of soybean expressing the single genes Pti5 or RLK2 or the combination of both genes (RLK2+Pti5) in comparison to non-transgenic wildtype soybean, with and without fungicide treatment (untreated: no fungicide treatment, A treatment: one fungicide treatment at the onset of ASR disease (˜35-40 days after planting)). The dotted bar shows the predicted relative yield increase based on the Colby Formula (see FIG. 2) when using the yield increases mediated by both single genes. If the dotted bar would be lower than the diagonally striped bar showing the real measured yield increase mediated by the combination of Pti5 and RLK2 (stack), the result could be considered more than additive. The graph shows the results measured at location 1.

It is clearly visible that the yield increase mediated by the combination of Pti5 and RLK2 is much lower than the additive yield increase that is predicted by the Colby formula based on the performance of the single genes. So, the combination of RLK2 and Pti5 leads to less than additive yields.

FIG. 5b shows the relative yield increase [%] of soybean expressing the single genes Pti5 or RLK2 or the combination of both genes (RLK2+Pti5) in comparison to non-transgenic wildtype soybean, with and without fungicide treatment (untreated: no fungicide treatment, A treatment: one fungicide treatment at the onset of ASR disease (˜35-40 days after planting)). The dotted bar shows the predicted relative yield increase based on the Colby Formula (see FIG. 2) when using the yield increases mediated by both single genes. If the dotted bar is lower than the diagonally striped bar showing the real measured yield increase mediated by the combination of Pti5 and RLK2 (stack), the result can be considered more than additive. The graph shows the results measured at location 2.

It is clearly visible that the yield increase mediated by the combination of Pti5 and RLK2 is much lower than the additive yield increase that is predicted by the Colby formula based on the performance of the single genes. So, the combination of RLK2 and Pti5 leads to less than additive yields.

FIG. 6a shows the relative yield increase [%] of soybean expressing the single genes SAR8.2 or RLK2 or the combination of both genes (RLK2+SAR8.2) in comparison to non-transgenic wildtype soybean, with and without fungicide treatment (untreated: no fungicide treatment, A treatment: one fungicide treatment at the onset of ASR disease (˜35-40 days after planting)). The dotted bar shows the predicted relative yield increase based on the Colby Formula (see FIG. 2) when using the yield increases mediated by both single genes. If the dotted bar would be lower than the diagonally striped bar showing the real measured yield increase mediated by the combination of SAR8.2 and RLK2 (stack), the result could be considered more than additive. The graph shows the results measured at location 1.

It is clearly visible that the yield increase mediated by the combination of SAR8.2 and RLK2 is much lower than the additive yield increase that is predicted by the Colby formula based on the performance of the single genes. So, the combination of RLK2 and SAR8.2 leads to less than additive yields.

FIG. 6b shows the relative yield increase [%] of soybean expressing the single genes SAR8.2 or RLK2 or the combination of both genes (RLK2+SAR8.2) in comparison to non-transgenic wildtype soybean, with and without fungicide treatment (untreated: no fungicide treatment, A treatment: one fungicide treatment at the onset of ASR disease (˜35-40 days after planting)). The dotted bar shows the predicted relative yield increase based on the Colby Formula (see FIG. 2) when using the yield increases mediated by both single genes. If the dotted bar is lower than the diagonally striped bar showing the real measured yield increase mediated by the combination of SAR8.2 and RLK2 (stack), the result can be considered more than additive. The graph shows the results measured at location 2.

It is clearly visible that the yield increase mediated by the combination of SAR8.2 and RLK2 is much lower than the additive yield increase that is predicted by the Colby formula based on the performance of the single genes. So, the combination of RLK2 and SAR8.2 leads to less than additive yields.

FIG. 7 shows the relative yield increase [%] of soybean expressing the single genes Pti5 or Ein2Cterm or the combination of both genes (Ein2Cterm+Pti5) in comparison to nontransgenic wildtype soybean, with and without fungicide treatment (untreated: no fungicide treatment, A treatment: one fungicide treatment at the onset of ASR disease (˜35-40 days after planting). The dotted bar shows the predicted relative yield increase based on the Colby Formula (see FIG. 2) when using the yield increases mediated by both single genes. If the dotted bar would be lower than the diagonally striped bar showing the real measured yield increase mediated by the combination of Pti5 and Ein2Cterm (stack), the result could be considered more than additive.

It is clearly visible that the yield increase mediated by the combination of Pti5 and Ein2Cterm is much lower than the additive yield increase that is predicted by the Colby formula based on the performance of the single genes. So, the combination of Ein2Cterm and Pti5 leads to less than additive yields.

FIG. 8 shows a scheme for replacing amino acids in the sequence of the Pti5 protein. The amino acid positions are given in chunks of at most 100 amino acids (here: 1-100 and 101-161). For each position, the number of asterisks denotes the degree of conservation, with a higher column of asterisks for a position indicating a higher preference to maintain the respective most preferred amino acid. The amino acid sequence below the rows of asterisks is the sequence of most preferred amino acids. The second amino acid sequence below the rows of asterisks is the sequence according to SEQ ID NO. 1. The columns of amino acids below the most preferred sequence indicate, for each position, the replacements preferred according to the invention, wherein the replacements are sorted in decreasing order of preference. Replacements are given by their standard 1-letter amino acid abbreviations, wherein ‘-’ indicates a missing amino acid such that, after alignment to the top sequence, a gap appears in the aligned sequence.

FIG. 9 shows a scheme for replacing amino acids in the sequence of the SAR8.2 protein. The amino acid positions are given in chunks of at most 100 amino acids (here: 1-86). For each position, the number of asterisks denotes the degree of conservation, with a higher column of asterisks for a position indicating a higher preference to maintain the respective most preferred amino acid. The amino acid sequence below the rows of asterisks is the sequence of most preferred amino acids. The second amino acid sequence below the rows of asterisks is the sequence according to SEQ ID NO. 2. The columns of amino acids below the most preferred sequence indicate, for each position, the replacements preferred according to the invention, wherein the replacements are sorted in decreasing order of preference. Replacements are given by their standard 1-letter amino acid abbreviations, wherein ‘-’ indicates a missing amino acid such that, after alignment to the top sequence, a gap appears in the aligned sequence.

| SEQ ID. | nt/aa | description |
|---|---|---|
| 1 | aa | artificial Pti5-like sequence |
| 2 | aa | artificial SAR8.2-like sequence |
| 3 | aa | Pti5 protein sequence |
| 4 | nt | DNA sequence coding for the Pti5 |
| | | protein of SEQ ID NO. 3 |
| 5 | aa | SAR8.2A protein sequence |
| 6 | nt | DNA sequence coding for the |
| | | SAR8.2A protein of SEQ ID NO. 5 |

### DETAILED DESCRIPTION OF THE INVENTION

The technical teaching of the invention is expressed herein using the means of language, in particular by use of scientific and technical terms. However, the skilled person understands that the means of language, detailed and precise as they may be, can only approximate the full content of the technical teaching, if only because there are multiple ways of expressing a teaching, each necessarily failing to completely express all conceptual connections, as each expression necessarily must come to an end. With this in mind the skilled person understands that the subject matter of the invention is the sum of the individual technical concepts signified herein or expressed, necessarily in a pars-pro-toto way, by the innate constrains of a written description. In particular, the skilled person will understand that the signification of individual technical concepts is done herein as an abbreviation of spelling out each possible combination of concepts as far as technically sensible, such that for example the disclosure of three concepts or embodiments A, B and C are a shorthand notation of the concepts A+B, A+C, B+C, A+B+C. In particular, fallback positions for features are described herein in terms of lists of converging alternatives or instantiations. Unless stated otherwise, the invention described herein comprises any combination of such alternatives. The choice of more or less preferred elements from such lists is part of the invention and is due to the skilled person's preference for a minimum degree of realization of the advantage or advantages conveyed by the respective features. Such multiple combined instantiations represent the adequately preferred form(s) of the invention.

In so far as recourse herein is made to entries in public databases, for example Uniprot and PFAM, the contents of these entries are those as of 2020-05-20. Unless stated to the contrary, where the entry comprises a nucleic acid or amino acid sequence information, such sequence information is incorporated herein.

As used herein, terms in the singular and the singular forms like “a”, “an” and “the” include plural referents unless the content clearly dictates otherwise. Thus, for example, use of the term “a nucleic acid” optionally includes, as a practical matter, many copies of that nucleic acid molecule; similarly, the term “probe” optionally (and typically) encompasses many similar or identical probe molecules. Also as used herein, the word “comprising” or variations such as “comprises” or “comprising” will be understood to imply the inclusion of a stated element, integer or step, or group of elements, integers or steps, but not the exclusion of any other element, integer or step, or group of elements, integers or steps.

As used herein, the term “and/or” refers to and encompasses any and all possible combinations of one or more of the associated listed items, as well as the lack of combinations when interpreted in the alternative (“or”). The term “comprising” also encompasses the term “consisting of”.

The term “about”, when used in reference to a measurable value, for example an amount of mass, dose, time, temperature, sequence identity and the like, refers to a variation of ±0.1%, 0.25%, 0.5%, 0.75%, 1%, 2%, 3%, 4%, 5%, 6%, 7%, 8%, 9%, 10%, 15% or even 20% of the specified value as well as the specified value. Thus, if a given composition is described as comprising “about 50% X,” it is to be understood that, in some embodiments, the composition comprises 50%×whilst in other embodiments it may comprise anywhere from 40% to 60%×(i.e., 50%±10%).

As used herein, the term “gene” refers to a biochemical information which, when materialized in a nucleic acid, can be transcribed into a gene product, i.e. a further nucleic acid, preferably an RNA, and preferably also can be translated into a peptide or polypeptide. The term is thus also used to indicate the section of a nucleic acid resembling said information and to the sequence of such nucleic acid (herein also termed “gene sequence”).

Also as used herein, the term “allele” refers to a variation of a gene characterized by one or more specific differences in the gene sequence compared to the wild type gene sequence, regardless of the presence of other sequence differences. Alleles or nucleotide sequence variants of the invention have at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% nucleotide “sequence identity” to the nucleotide sequence of the wild type gene. Correspondingly, where an “allele” refers to the biochemical information for expressing a peptide or polypeptide, the respective nucleic acid sequence of the allele has at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% amino acid “sequence identity” to the respective wild type peptide or polypeptide.

Protein or nucleic acid variants may be defined by their sequence identity when compared to a parent protein or nucleic acid. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A:
AAGATACTG length: 9 bases
Seq B:
GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “I” symbol in the alignment indicates identical residues (which means bases for DNA or amino acids for proteins). The number of identical residues is 6.

The “-” symbol in the alignment indicates gaps. The number of gaps introduced by alignment within the sequence B is 1. The number of gaps introduced by alignment at borders of sequence B is 2, and at borders of sequence A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing sequence A over its complete length would be 9 (meaning sequence A is the sequence of the invention), the alignment length showing sequence B over its complete length would be 8 (meaning sequence B is the sequence of the invention).

After aligning the two sequences, in a second step, an identity value shall be determined from the alignment. Therefore, according to the present description the following calculation of percent-identity applies:
%-identity=(identical residues/length of the alignment region which is showing the respective sequence of this invention over its complete length)*100. Thus, sequence identity in relation to comparison of two amino acid sequences according to the invention is calculated by dividing the number of identical residues by the length of the alignment region which is showing the respective sequence of this invention over its complete length. This value is multiplied with 100 to give “%-identity”. According to the example provided above, %-identity is: for sequence A being the sequence of the invention (6/9)*100=66.7%; for sequence B being the sequence of the invention (6/8)*100=75%.

The term “nucleic acid construct” as used herein refers to a nucleic acid molecule, either singleor double-stranded, which is isolated from a naturally occurring gene or is modified to contain segments of nucleic acids in a manner that would not otherwise exist in nature or is synthetic.

The term “nucleic acid construct” is synonymous with the term “expression cassette” when the nucleic acid construct contains the control sequences required for expression of a polynucleotide.

The term “control sequence” or “genetic control element” is defined herein to include all sequences affecting the expression of a polynucleotide, including but not limited thereto, the expression of a polynucleotide encoding a polypeptide. Each control sequence may be native or foreign to the polynucleotide or native or foreign to each other. Such control sequences include, but are not limited to, promoter sequence, 5′-UTR (also called leader sequence), ribosomal binding site (RBS), 3′-UTR, and transcription start and stop sites.

The term “functional linkage” or “operably linked” with respect to regulatory elements is to be understood as meaning the sequential arrangement of a regulatory element (including but not limited thereto a promoter) with a nucleic acid sequence to be expressed and, if appropriate, further regulatory elements (including but not limited thereto a terminator) in such a way that each of the regulatory elements can fulfil its intended function to allow, modify, facilitate or otherwise influence expression of said nucleic acid sequence. For example, a control sequence is placed at an appropriate position relative to the coding sequence of the polynucleotide sequence such that the control sequence directs the expression of the coding sequence of a polypeptide.

A “promoter” or “promoter sequence” is a nucleotide sequence located upstream of a gene on the same strand as the gene that enables that gene's transcription. A promoter is generally followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

As used herein, the term “isolated DNA molecule” refers to a DNA molecule at least partially separated from other molecules normally associated with it in its native or natural state. The term “isolated” preferably refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

Any number of methods well known to those skilled in the art can be used to isolate and manipulate a polynucleotide, or fragment thereof, as disclosed herein. For example, polymerase chain reaction (PCR) technology can be used to amplify a particular starting polynucleotide molecule and/or to produce variants of the original molecule. Polynucleotide molecules, or fragment thereof, can also be obtained by other techniques, such as by directly synthesizing the fragment by chemical means, as is commonly practiced by using an automated oligonucleotide synthesizer. A polynucleotide can be single-stranded (ss) or double-stranded (ds). “Double-stranded” refers to the base-pairing that occurs between sufficiently complementary, anti-parallel nucleic acid strands to form a double-stranded nucleic acid structure, generally under physiologically relevant conditions. Embodiments of the method include those wherein the polynucleotide is at least one selected from the group consisting of sense single-stranded DNA (ssDNA), sense single-stranded RNA (ssRNA), double-stranded RNA (dsRNA), double-stranded DNA (dsDNA), a double-stranded DNA/RNA hybrid, anti-sense ssDNA, or anti-sense ssRNA; a mixture of polynucleotides of any of these types can be used.

As used herein, “recombinant” when referring to nucleic acid or polypeptide, indicates that such material has been altered as a result of human application of a recombinant technique, such as by polynucleotide restriction and ligation, by polynucleotide overlap-extension, or by genomic insertion or transformation. A gene sequence open reading frame is recombinant if (a) that nucleotide sequence is present in a context other than its natural one, for example by virtue of being (i) cloned into any type of artificial nucleic acid vector or (ii) moved or copied to another location of the original genome, or if (b) the nucleotide sequence is mutagenized such that it differs from the wild type sequence. The term recombinant also can refer to an organism having a recombinant material, e.g., a plant that comprises a recombinant nucleic acid is a recombinant plant.

The term “transgenic” refers to an organism, preferably a plant or part thereof, or a nucleic acid that comprises a heterologous polynucleotide. Preferably, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been so altered by the presence of heterologous nucleic acid including those transgenic organisms or cells initially so altered, as well as those created by crosses or asexual propagation from the initial transgenic organism or cell. A “recombinant” organism preferably is a “transgenic” organism. The term “transgenic” as used herein is not intended to encompass the alteration of the genome (chromosomal or extra-chromosomal) by conventional plant breeding methods (e.g., crosses) or by naturally occurring events such as, e.g., self-fertilization, random cross-fertilization, nonrecombinant viral infection, non-recombinant bacterial transformation, non-recombinant transposition, or spontaneous mutation.

As used herein, “mutagenized” refers to an organism or nucleic acid thereof having alteration(s) in the biomolecular sequence of its native genetic material as compared to the sequence of the genetic material of a corresponding wildtype organism or nucleic acid, wherein the alteration(s) in genetic material were induced and/or selected by human action. Examples of human action that can be used to produce a mutagenized organism or DNA include, but are not limited to treatment with a chemical mutagen such as EMS and subsequent selection with herbicide(s); or by treatment of plant cells with x-rays and subsequent selection with herbicide(s). Any method known in the art can be used to induce mutations. Methods of inducing mutations can induce mutations in random positions in the genetic material or can induce mutations in specific locations in the genetic material (i.e., can be directed mutagenesis techniques), such as by use of a genoplasty technique. In addition to unspecific mutations, according to the invention a nucleic acid can also be mutagenized by using mutagenesis means with a preference or even specificity for a particular site, thereby creating an artificially induced heritable allele according to the present invention. Such means, for example site specific nucleases, including for example zinc finger nucleases (ZFNs), meganucleases, transcription activator-like effector nucleases (TALENS) (Malzahn et al., Cell Biosci, 2017, 7:21) and clustered regularly interspaced short palindromic repeats/CRISPR-associated nuclease (CRISPR/Cas) with an engineered crRNA/tracr RNA (for example as a single-guide RNA, or as modified crRNA and tracrRNA molecules which form a dual molecule guide), and methods of using this nucleases to target known genomic locations, are well known in the art (see reviews by Bortesi and Fischer, 2015, Biotechnology Advances 33: 41-52; and by Chen and Gao, 2014, Plant Cell Rep 33: 575-583, and references within).

As used herein, a “genetically modified organism” (GMO) is an organism whose genetic characteristics contain alteration(s) that were produced by human effort causing transfection that results in transformation of a target organism with genetic material from another or “source” organism, or with synthetic or modified-native genetic material, or an organism that is a descendant thereof that retains the inserted genetic material. The source organism can be of a different type of organism (e.g., a GMO plant can contain bacterial genetic material) or from the same type of organism (e.g., a GMO plant can contain genetic material from another plant).

As used herein, “wildtype” or “corresponding wildtype plant” means the typical form of an organism or its genetic material, as it normally occurs, as distinguished from e.g. mutagenized and/or recombinant forms. Similarly, by “control cell”, “wildtype” “control plant, plant tissue, plant cell or host cell” is intended a plant, plant tissue, plant cell, or host cell, respectively, that lacks the particular polynucleotide of the invention that are disclosed herein. The use of the term “wildtype” is not, therefore, intended to imply that a plant, plant tissue, plant cell, or other host cell lacks recombinant DNA in its genome, and/or does not possess fungal resistance characteristics that are different from those disclosed herein.

As used herein, “descendant” refers to any generation plant. A progeny or descendant plant can be from any filial generation, e.g., F1, F2, F3, F4, F5, F6, F7, etc. In some embodiments, a descendant or progeny plant is a first, second, third, fourth, fifth, sixth, seventh, eighth, ninth, or tenth generation plant.

The term “plant” is used herein in its broadest sense as it pertains to organic material and is intended to encompass eukaryotic organisms that are members of the taxonomic kingdom plantae, examples of which include but are not limited to monocotyledon and dicotyledon plants, vascular plants, vegetables, grains, flowers, trees, herbs, bushes, grasses, vines, ferns, mosses, fungi and algae, etc, as well as clones, offsets, and parts of plants used for asexual propagation (e.g. cuttings, pipings, shoots, rhizomes, underground stems, clumps, crowns, bulbs, corms, tubers, rhizomes, plants/tissues produced in tissue culture, etc.). Unless stated otherwise, the term “plant” refers to a whole plant, any part thereof, or a cell or tissue culture derived from a plant, comprising any of: whole plants, plant components or organs (e.g., leaves, stems, roots, etc.), plant tissues, seeds, plant cells, and/or progeny of the same. A plant cell is a biological cell of a plant, taken from a plant or derived through culture from a cell taken from a plant.

The invention particularly applies to plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to a preferred embodiment of the present invention, the plant is a crop plant. Examples of crop plants include inter alia soybean, sunflower, canola, alfalfa, rapeseed, cotton, tomato, potato or tobacco. The plant preferably is not of taxonomic family Solanaceae, more preferably not of sub-family Solanoidae. Most preferably the plant is of genus Glycine as described herein.

According to the invention, a plant is cultivated to yield plant material. Cultivation conditions are chosen in view of the plant and may include, for example, any of growth in a greenhouse, growth on a field, growth in hydroculture and hydroponic growth.

The plant, hereinafter also called “yield improvement plant”, preferably comprises a Pti5 and a SAR8.2 gene, preferably each in its own expression cassette as explained below. It has now surprisingly been found that these genes, when combined in one plant cell, can convey improved yield, preferably even super-additive yield improvement (herein: “synergistic” yield improvement). It is noteworthy that the preferably synergistic yield improvement is both under standard growth conditions established in the respective region of field plantation and under pathogen challenged growth conditions, in particular under fungal pathogen prevalence in the region where the plants are grown on fields. According to the present invention it is preferred that pathogen pressure is determined according to the average diseased leaf area of plants and is preferably expressed as the area under disease-progression curve.

As plants are grown by cell division, references herein to plants comprising one or more Pti5 genes and one or more SAR8.2 genes and/or at least one Pti5-SAR8.2 fusion gene (hereinafter collectively named “Pti5-SAR8.2 combination” or “stack”) always also refer to (1) one or more cells comprising a nucleic acid coding for a Pti5-SAR8.2 stack and (2) to plant parts, in particular organs, preferably leaves, of such plants comprising such cells.

Plants comprising either a Pti5 or SAR8.2 gene have been described before, among others, in WO2013001435 and WO2014076614. The documents, however, does not show any improvement of yield. Instead, they focus on the achievement of fungal resistance. As shown herein, however, fungal resistance is no predictor for yield improvements. Thus, these documents only provide a general technical background concerning certain plants comprising the aforementioned genes but do not imply or even render likely that any yield improvement as described by the present invention can be achieved.

For the purposes of the invention, a Pti5 gene codes for a protein comprising, among others, an apetala 2 domain as explained in PFAM entry PF00847 and binding to the Pti5 GCC box as described by Gu et al 2002 The Plant Cell, Vol. 14, 817-831. Preferably, the Pti5 gene codes for a protein whose amino acid sequence has at least 40%, more preferably at least 43%, more preferably at least 50%, more preferably at least 58%, more preferably at least 67%, more preferably at least 70%, more preferably at least 71% sequence identity to SEQ ID NO. 1, wherein preferably the sequence identity to SEQ ID NO. 1 is at most 80%, more preferably at most 79%. Particularly preferred are thus plants expressing a Pti5 gene whose corresponding polypeptide sequence has 58-80% sequence identity to SEQ ID NO. 1, more preferably 67-79% sequence identity to SEQ ID NO. 1. It is to be understood that SEQ ID NO. 1 is an artificial amino acid sequence specifically constructed as a template for amino acid sequence annealing purposes. The sequence can thus be used for identification of Pti5 genes independent from the fact that no Pti5 activity of the polypeptide of SEQ ID NO. 1 is shown herein. Particularly preferred as a

Pti5 gene in a method or plant according to the present invention is any of the amino acid sequences defined by the following Uniprot identifiers: PTI5_SOLLC, M1AQ94_SOLTU, A0A2G3A6U8_CAPAN, A0A2G2XEI7_CAPBA, A0A2G3D5K5_CAPCH, A0A1S4BF73_TOBAC, A0A 1U7WC00_NICSY, A0A1S4A5G9_TOBAC, A0A 1J6J1M1_NICAT, A0A1S2X9U7_CICAR, G7IFJO_MEDTR, A0A2K3KXT4_TRIPR, V7BQ20_PHAVU, A0A1S3VIX3_VIGRR, AOAOL9VF85_PHAAN, A0A445GQU3_GLYSO, AOAOROG4Q5_SOYBN, A0A061GM02_THECC, A0A44518U7_GLYSO, AOAOD2S2G5_GOSRA, A0A4P1QVV4_LUPAN, A0A151SAR8.21_CAJCA, A0A2J6MBZ7_LACSA, A0A2K3LDZ4_TRIPR, A0A2U1QDE9_ARTAN, A0A444WYK6_ARAHY. Particularly preferred according to the invention are Pti5 genes, and plants expressing them, which code for a polypeptide having at least 60%, more preferably at least 71%, more preferably at least 75%, more preferably at least 79%, more preferably at least 82%, more preferably at least 90% more preferably at least 95% sequence identity to the amino acid sequence given by Uniprot identifier PTI5_SOLLC and preferably differs from this sequence by 0-20 amino acids, more preferably 1-15 amino acids, even more preferably 1-10 amino acids, even more preferably 1-5 amino acids. Preferably deviations from the Pti5 protein sequence conform to the constraints according to FIG. 8. If the Pti5 sequence, when aligned to the sequence according to Uniprot identifier PTI5_SOLLC, is longer than said sequence, then each C- or N-terminal extension is preferably no longer than 10 amino acids, more preferably 0-5 amino acids.

For the purposes of the invention, a SAR8.2 gene codes for a protein comprising or consisting of a SAR8.2 domain as explained in PFAM entry PF03058. Preferably, the SAR8.2 gene codes for a protein whose amino acid sequence has at least 35%, more preferably at least 45%, more preferably at least 55%, more preferably at least 72%, more preferably at least 77%, more preferably at least 82%, more preferably at least 84%, more preferably at least 86%, more preferably at least 88%, more preferably at least 89% sequence identity to SEQ ID NO. 2, wherein preferably the sequence identity to SEQ ID NO. 2 is at most 98%, more preferably at most 95%. Particularly preferred are thus plants expressing a SAR8.2 gene whose corresponding polypeptide sequence has 72-98% sequence identity to SEQ ID NO. 2, more preferably 74-92% sequence identity to SEQ ID NO. 2. It is to be understood that SEQ ID NO. 2 is an artificial amino acid sequence specifically constructed as a template for amino acid sequence annealing purposes. The sequence can thus be used for identification of SAR8.2 genes independent from the fact that no SAR8.2 activity of the polypeptide of SEQ ID NO. 2 is shown herein. Particularly preferred as a SAR8.2 gene in a method or plant according to the present invention is any of the amino acid sequences defined by the following Uniprot identifiers: Q8W2C1_CAPAN, Q9SEM2_CAPAN, A0A2G2X990_CAPBA, Q947G6_CAPAN, Q947G5_CAPAN, A0A2G2X9U8_CAPBA, A0A2G3CEJ1_CAPCH, A0A2G2X931_CAPBA, M1BEK3_SOLTU, A0A3Q7J4M2_SOLLC, A0A2G2ZTB6_CAPAN, A0A2G3CRF6_CAPCH, A0A2G2W296_CAPBA, A0A2G2WZ87_CAPBA, M1BIQ9_SOLTU, M1D489_SOLTU, M1D488_SOLTU, A0A2G2ZQ02_CAPAN, A0A1S4AM24_TOBAC, A0A1U7XJ42_NICSY, A0A1S4CJX7_TOBAC. Particularly preferred according to the invention are SAR8.2 genes, and plants expressing them, which code for a polypeptide having at least 60%, more preferably at least 68%, more preferably at least 88%, more preferably at least 91%, more preferably at least 95% sequence identity to the amino acid sequence given by Uniprot identifier Q8W2C1_CAPAN and preferably differs from this sequence by 0-20 amino acids, more preferably 1-15 amino acids, even more preferably 1-10 amino acids, even more preferably 1-5 amino acids. Preferably deviations from the Pti5 protein sequence conform to the constraints according to FIG. 9. If the SAR8.2 sequence, when aligned to the sequence according to Uniprot identifier Q8W2C1_CAPAN, is longer than said sequence, then each C- or N-terminal extension is preferably no longer than 10 amino acids, more preferably 0-5 amino acids.

Preferred according to the invention are cells, particularly plant cells or plant cell containing plant parts or whole plants which comprise
a) a gene coding for a polypeptide which has at least 60%, more preferably at least 71%, more preferably at least 75%, more preferably at least 79%, more preferably at least 82%, more preferably at least 90%, even more preferably at least 95% sequence identity to the amino acid sequence given by Uniprot identifier PTI5_SOLLC and preferably differs from this sequence by 0-20 amino acids, more preferably 1-15 amino acids, even more preferably 1-10 amino acids, even more preferably 1-5 amino acids, according to the constraints given in FIG. 8, and
b) a gene coding for a polypeptide which has at least 60%, more preferably at least 68%, more preferably at least 88%, more preferably at least 91%, more preferably at least 95% sequence identity to the amino acid sequence given by Uniprot identifier Q8W2C1_CAPAN and preferably differs from this sequence by 0-20 amino acids, more preferably 1-15 amino acids, even more preferably 1-10 amino acids, even more preferably 1-5 amino acids, according to the constraints given in FIG. 9.

Even more preferred according to the invention are cells, particularly plant cells or plant cell containing plant parts or whole plants which comprise
a) a gene coding for a polypeptide which has at least 79%, more preferably at least 82%, more preferably at least 90%, even more preferably at least 95% sequence identity to the amino acid sequence given by Uniprot identifier PTI5_SOLLC and preferably differs from this sequence by 0-20 amino acids, more preferably 1-15 amino acids, even more preferably 1-10 amino acids, even more preferably 1-5 amino acids, according to the constraints given in FIG. 8, and
b) a gene coding for a polypeptide which has at least 88%, more preferably at least 91%, more preferably at least 95% sequence identity to the amino acid sequence given by Uniprot identifier Q8W2C1_CAPAN and preferably differs from this sequence by 0-20 amino acids, more preferably 1-15 amino acids, even more preferably 1-10 amino acids, even more preferably 1-5 amino acids according to the constraints given in FIG. 9.

Expression of the Pti5 and SAR8.2 protein can be effected in the cell by transcription and translation from a Pti5 gene and a SAR8.2 gene separated from the Pti5 gene by at least 1 stop codon. The Pti5 and SAR8.2 genes can be contained in a single expression cassette. Preferably the genes coding for Pti5 and SAR8.2 are contained in the cell in separate expression cassettes as described herein.

Furthermore, expression of the Pti5 and SAR8.2 protein can be effected by transcription and translation from a Pti5-SAR8.2 fusion gene which codes for a Pti5-SAR8.2 fusion protein. In such fusion protein the sections coding for the Pti5 moiety and the SAR8.2 moiety are linked by a linker sequence. Preferably the linker sequence codes for a linker of 1-30 amino acids, more preferably 1-20 amino acids. Preferably the linker sequence comprises a protease cleavage site operable in the cell. Thus, during expression of the fusion gene in cells of the plant of the present invention the pre-protein resulting from transcription and translation of the fusion gene is cleaved to release the mature Pti5 protein and the mature SAR8.2 protein. In case of such fusion protein, the degree of sequence identity described above is determined on the basis of the mature Pti5 and SAR8.2 protein, respectively. In a fusion protein the sequence of the Pti5 and SAR8.2 moieties on the respective mRNA is of no particular concern. Thus, a fusion protein can comprise, in C-to-N-direction, a Pti5 moiety contiguous to a linker contiguous to a SAR8.2 moiety, or, in C-to-N-direction, a SAR8.2 moiety contiguous to a linker contiguous to a Pti5 moiety. As the mode of Pti5 and SAR8.2 protein generation is of no concern, all references according to the invention to a combination of Pti5 and SAR8.2 genes or proteins implicitly also encompass a Pti5-SAR8.2 fusion gene, and references to a set of Pti5 and SAR8.2 proteins also implicitly encompass a set of mature Pti5 and SAR8.2 proteins originating from cleavage of a Pti5-SAR8.2 fusion protein.

According to the present invention, the cell, plant part or plant preferably comprises an expression cassette for the Pti5 gene and an expression cassette for the SAR8.2 gene. According to the invention, an expression cassette comprises the respective gene—or fusion gene—and the control sequences required for expression of the gene. Preferably an expression cassette comprises at least a promoter and, operably linked thereto, the respective gene selected from Pti5 and SAR8.2. More preferably, the expression cassette also comprises a terminator in 3′ direction downstream of the respective gene. Exemplary expression cassettes for individual Pti5 and SAR8.2 genes are disclosed, for example, in the aforementioned documents WO2013001435 and WO2014076614, in particular those comprising the sequences SEQ ID NO. 6 and 3, respectively. Those expression cassettes and corresponding description are incorporated herein by reference.

Each of the Pti5 and SAR8.2 expression cassettes preferably is a heterologous expression cassette. According to the invention, the expression cassette is “heterologous” if one or more of the following conditions is fulfilled: (1) The gene codes for a polypeptide (Pti5 or SAR8.2, respectively) with a sequence different to the wild type plant; (2) the gene is under control of a promoter not present in the wild type plant or not connected to the gene in the wild type plant; (3) the expression cassette is integrated at a different locus in the plant genome compared to the wild type plant, wherein the wild type expression cassette can be in an inactivated form, or the heterologously integrated expression cassette is in addition to the wild type expression cassette. Thus, the yield improvement plants used according to the present invention preferably are transgenic plants. Furthermore, the methods according to the present invention preferably exclude plants exclusively obtained by means of an essentially biological process, e.g. the crossing of gametes found in nature. This preferred exclusion has no technical reason but is exclusively intended to form a basis for an amendment of the claims in those countries where the exclusion is mandatory. Preferably not excluded, however, are plants obtained by crossing and selection of at least one transgenic plant and another plant, as long as the offspring comprises both Pti5 and SAR8.2 genes (and/or a Pti5-SAR8.2 fusion gene), wherein preferably the Pti5 or SAR8.2 gene is present in the offspring in the form of a heterologous expression cassette, regardless if the offspring also contains a wild type Pti5 or SAR8.2 expression cassette. Most preferably the offspring contains a heterologous Pti5 and a heterologous SAR8.2 expression cassette, and even more preferably does not contain a wild type Pti5 and SAR8.2 gene.

According to the present invention, the cell, plant part or plant preferably comprises a wild type Pti5 expression cassette and a heterologous SAR8.2 expression cassette independent of the presence of a wild-type SAR8.2 expression cassette. Alternatively but also preferred the plant comprises a wild type SAR8.2 expression cassette and a heterologous Pti5 expression cassette independent of the presence of a wild-type Pti5 expression cassette. More preferred the plant comprises a wild type Pti5 expression cassette and a heterologous SAR8.2 expression cassette and lacks a functional wild-type SAR8.2 expression cassette, or the plant comprises a wild type SAR8.2 expression cassette and a heterologous Pti5 expression cassette and lacks a functional wild-type Pti5 expression cassette. Even more preferred the plant comprises a heterologous Pti5 expression cassette and a heterologous SAR8.2 expression cassette independent of the presence of a wild-type Pti5 expression cassette and also independent of the presence of a wild-type SAR8.2 expression cassette. Most preferably the plant comprises (1) a heterologous Pti5 expression cassette and a heterologous SAR8.2 expression cassette and/or (2) a Pti5-SAR8.2 fusion gene expression cassette, and lacks both a functional wild-type Pti5 expression cassette and a functional wild-type SAR8.2 expression cassette.

A heterologous Pti5, SAR8.2 or Pti5-SAR8.2 fusion expression cassette, respectively, can be introduced into a plant cell using a vector comprising only one of the aforementioned expression cassettes, two of the aforementioned expression cassettes or even three of the aforementioned expression cassettes. Preferably the vector comprises 1 expression cassette for expression of a Pti5 protein and 1 expression cassette for expression of a SAR8.2 gene. Preferably, the Pti5 and SAR8.2 protein are encoded by separate expression cassettes. When these separate expression cassettes are located on a single vector, then they are oriented in head-to-tail, head-to-head or tail-to-tail direction.

Heterologous Pti5 and SAR8.2 expression cassettes can also be introduced by transforming a plant cell using two separate vectors, wherein one vector does not comprise a SAR8.2 expression cassette and the other vector does not comprise a Pti5 expression cassette. Transformation by separate vectors can be done by co-transformation or super-transformation. In a cotransformation both genes would be located on 2 different T-DNAs, either in one or different Agrobacterium strains that are used for transformation. When using super-transformation a plant cell that already contains one of the genes is subsequently transformed with the second gene.

Plant cells capable of expressing both Pti5 and SAR8.2 can also be prepared by crossing parent plants, wherein one parent plant comprises at least a Pti5 expression cassette and the other parent plant comprises at least a SAR8.2 expression cassette, and selection of those offspring comprising both a Pti5 and a SAR8.2 expression cassette. The resulting F1 generation will contain both genes in a hemizygous way. Further selfing would lead to plants containing both expression cassettes homozygously and therefore fixed for subsequent generations.

According to the invention the plants-hybrid, homozygous, heterozygous or hemizygous with respect to the Pti5 and SAR8.2 gene or fusion gene as such—are grown under appropriate conditions. Growth of the plants according to the present invention leads to improved yield particularly under in-field growth conditions in contrast to sheltered greenhouse conditions. As shown in the examples it is particularly striking that yield improvements can be obtained in a synergistic, super-additive way under pathogen pressure even with minimal or without pesticide treatment. It is a particular advantage of the present invention that the plants can be cultivated using any of the applicable cultivation techniques established in the art. Thus, the invention advantageously provides methods applicable under the broadest variety of cultivation conditions including growth on a field and in a greenhouse. Thus, the use of the Pti5 and SAR8.2 gene combination to improve yield under all pathogen pressure conditions is surprisingly versatile.

According to the invention the yield is preferably one or more of
biomass per area of plantation,
grain mass per area of plantation,
seed mass per area of plantation,
   the last alternative being the most preferred definition of yield.

As used herein, “yield” refers to the amount of agricultural production harvested per unit of land. Yield can be any of total harvested biomass per area, total harvested grain mass per area and total harvested seed mass per area. Yield is measured by any unit, for example metric ton per hectare or bushels per acre. Yield is adjusted for moisture of harvested material, wherein moisture is measured at harvest in the harvested biomass, grain or seed, respectively. For example, moisture of soybean seed is preferably 15%.

As described above, yield improvement is measured in comparison to the yield obtained by a control plant. The control plant is a plant lacking the expression cassettes referred to above, but is otherwise cultivated under identical conditions. Improvement of yield is determined by the yield of a “yield improvement plant” comprising said heterologous expression cassettes relative to a control plant of the same species or, if applicable, variety, wherein the control plant does not comprise said heterologous expression cassette.

It is to be understood that when reference is made to yield or growing or cultivation or treatment of “a plant”, it is preferred not to determine the yield or perform the treatment on a single plant compared to a single control plant. Instead, yield is determined by the yield obtained from an ensemble of the plants, preferably an ensemble of at least 1000 plants, preferably wherein the plants are cultivated on a field or, less preferably, in a greenhouse. Most preferably the yield of a monoculture field of at least 1 ha of the plant and a monoculture field of at least 1 ha of the control plant is determined, respectively. Correspondingly, treatments are preferably performed on such ensemble of plants. It is a particular advantage that the use of the combination of at least one Pti5 and at least one SAR8.2 gene allows to secure yield improvements, compared to a non-transgenic wild type control plant, of at least 10%. More preferably, yield increase is synergistic, that is, it is more than the added yield changes caused individually by the Pti5 and SAR8.2 gene, wherein each Pti5 and SAR8.2 gene induced yield (preferably seed mass yield) change is measured in comparison against a respective control plant without a respective Pti5 or SAR8.2 expression cassette. It is a particular advantage of the present invention—also shown in particular in the examples below—that a yield increase of at least 10%, more preferably a synergistic yield increase, is obtainable even without pesticide treatment, preferably without fungicide treatment, but also is obtainable if plants are intermittently treated with one or more pesticides, preferably one or more fungicides, during a growth season from seed to harvest.

In view of the above advantages the invention also provides a farming method for improving the yield produced by a plant relative to a control plant, comprising cultivation of a plant comprising a Pti5 and a SAR8.2 gene, wherein during cultivation of the plant the number of pesticide treatments per growth season is reduced by at least one relative to the control plant, preferably by at least two. Preferably the farming method comprises growing a plant (a) overexpressing Pti5 and SAR8.2 and/or (b) comprising a heterologous Pti5 expression cassette and/or a heterologous SAR8.2 expression cassette and/or (c) expressing a heterologous Pti5 and/or heterologous SAR8.2 gene and/or (d) expressing a Pti5-SAR8.2 fusion gene. Pesticide treatment schemes are generally established in standard agricultural practice for each region of plant growth. For example, in Brazil it may be customary to apply a first fungicide treatment to soybean plants on day 8 after seeding and a second spray on day 18 after seeding. In other regions a scheme may be practiced not depending on mere time of growth but, for example, taking into account first notice of a pest occurrence or passing of a pest incidence threshold. It is a particular and unforeseen advantage of the present invention that the number of pesticide treatments per growth seasons can be reduced compared to a control plant. It was in particular surprising that such treatment reduction is possible not only without reducing yield; instead the farming method according to the invention advantageously allows to maintain or even increase yield despite the reduction in treatments. This greatly improves cost efficiency of farming the plants as provided by the present invention. Thus, the invention provides the farming methods or methods for yield improvement described herein, wherein preferably at most two fungicide treatments are applied in a growing season, that is, in the period between seeding and harvesting, more preferably at most one fungicide treatment is applied in a growing season. In suitable conditions the methods are performed without fungicide treatments in a growing season. Of course the pesticide is preferably applied in pesticidally effective amounts.

According to the invention the methods provided herein preferably provide an increased yield, relative to a control plant, in the absence or, more preferably, in the presence of a pathogen (also called “pest” herein). It is a particular advantage that the yield increase according to the invention not only can be achieved in a particular variety of climate conditions conductive for plant cultivation; the yield increase according to the invention has also consistently been found under most conditions. According to the invention the trait “yield improvement” is thus remarkably resilient under pest stress conditions. According to the invention, stress factors other than pest induced stress are preferably taken care of by established cultivation techniques. For example, nitrogen starvation stress is preferably removed by fertilization, and water limitation stress is preferably alleviated by irrigation.

According to the invention the pest preferably is or comprises at least a fungal pest, preferably a biotrophic or heminecrotrophic fungus, more preferably a rust fungus. If during cultivation the plant is also under threat of stress by other pathogens, e.g. nematodes and insects, such other pests are preferably taken care of by respective pesticide treatments. Thus, according to the invention preferably the number of fungicide treatments is reduced as described above, irrespective of other pesticide treatments. The fungicide is preferably applied in fungicidally effective amounts. The fungicide can be mixed with other pesticides and ingredients preferably selected from insecticides, nematicides, and acaricides, herbicides, plant growth regulators, fertilizers. Preferred mixing partners are insecticides, nematicides and fungicides. It is particularly preferred to reduce, during cultivation of the plant, the number of fungicide treatments per growth season by at least one relative to the control plant, preferably by at least two. Fungicides may include 2-(thiocyanatomethylthio)-benzothiazole, 2-phenylphenol, 8-hydroxyquinoline sulfate, ametoctradin, amisulbrom, antimycin, Ampelomyces quisqualis, azaconazole, azoxystrobin, Bacillus subtilis, Bacillus subtilis strain QST713, benalaxyl, benomyl, benthiavalicarb-isopropyl, benzylaminobenzene-sulfonate (BABS) salt, bicarbonates, biphenyl, bismerthiazol, bitertanol, bixafen, blasticidin-S, borax, Bordeaux mixture, boscalid, bromuconazole, bupirimate, calcium polysulfide, captafol, captan, carbendazim, carboxin, carpropamid, carvone, chlazafenone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenoconazole, difenzoquat ion, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, enestrobin, enestroburin, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fenpyrazamine, fentin, fentin acetate, fentin hydroxide, ferbam, ferimzone, fluazinam, fludioxonil, fluindapyr, flumorph, fluopicolide, fluopyram, fluoroimide, fluoxastrobin, fluquinconazole, flusilazole, flusulfamide, flutianil, flutolanil, flutriafol, fluxapyroxad, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), iodocarb, ipconazole, ipfenpyrazolone, iprobenfos, iprodione, iprovalicarb, isoprothiolane, isofetamide, isopyrazam, isotianil, kasugamycin, kasugamycin hydrochloride hydrate, kresoxim-methyl, laminarin, mancopper, mancozeb, mandipropamid, maneb, mefenoxam, mepanipyrim, mepronil, meptyldinocap, mercuric chloride, mercuric oxide, mercurous chloride, metalaxyl, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxathiapiprolin, oxine-copper, oxpoconazole fumarate, oxycarboxin, pefurazoate, penconazole, pencycuron, penflufen, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, pydiflumetofen, prothioconazole, pyraclostrobin, pyrametostrobin, pyraoxystrobin, pyraziflumid, pyrazophos, pyribencarb, pyributicarb, pyrifenox, pyrimethanil, pyriofenone, pyroquilon, quinoclamine, quinoxyfen, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxysporum, Gliocladium spp., Phlebiopsis gigantea, Streptomyces griseoviridis, Trichoderma spp., (RS)—N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-I-enyl)phenyl thiocyanateme, aminopyrifen, ampropylfos, anilazine, azithiram, barium polysulfide, Bayer 32394, benodanil, benquinox, bentaluron, benzamacril; benzamacril-isobutyl, benzamorf, benzovindiflupyr, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, copper bis(3-phenylsalicylate), copper zinc chromate, coumoxystrobin, cufraneb, cupric hydrazinium sulfate, cuprobam, cyclafuramid, cypendazole, cyprofuram, decafentin, dichlobentiazox, dichlone, dichlozoline, diclobutrazol, dimethirimol, dinocton, dinosulfon, dinoterbon, dipymetitrone, dipyrithione, ditalimfos, dodicin, drazoxolon, EBP, enoxastrobin, ESBP, etaconazole, etem, ethirim, fenaminosulf, fenaminstrobin, fenapanil, fenitropan, fenpicoxamid, fluindapyr, fluopimomide, fluotrimazole, flufenoxystrobin, furcarbanil, furconazole, furconazole-cis, furmecyclox, furophanate, glyodine, griseofulvin, halacrinate, Hercules 3944, hexylthiofos, ICIA0858, inpyrfluxam, ipfentrifluconazole, ipflufenoquin, isofetamid, isoflucypram, isopamphos, isovaledione, mandestrobin, mebenil, mecarbinzid, mefentrifluconazole, metazoxolon, methfuroxam, methylmercury dicyandiamide, metsulfovax, metyltetraprole, milneb, mucochloric anhydride, myclozolin, N-3,5-dichlorophenyl-succinimide, N-3-nitrophenylitaconimide, natamycin, Nethylmercurio-4-toluenesulfonanilide, nickel bis(dimethyldithiocarbamate), OCH, oxathiapiprolin, phenylmercury dimethyldithiocarbamate, phenylmercury nitrate, phosdiphen, picarbutrazox, prothiocarb; prothiocarb hydrochloride, pydiflumetofen, pyracarbolid, pyrapropoyne, pyraziflumid, pyridachlometyl, pyridinitril, pyrisoxazole, pyroxychlor, pyroxyfur, quinacetol, quinacetol sulfate, quinazamid, quinconazole, quinofumelin, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, triclopyricarb, triflumezopyrim, urbacid, zarilamid, and any combinations thereof.

The pathogen according to the invention preferably is a fungus or a fungus-like organism from the phyla Ascomycota, Basisiomycota or Oomycota, more preferably of phylum Basidiomycota, even more preferably of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae, even more preferably of genus Rhizoctonia, Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis, even more preferably of species Rhizoctonia alpina, Rhizoctonia bicornis, Rhizoctonia butinii, Rhizoctonia callae, Rhizoctonia carotae, Rhizoctonia endophytica, Rhizoctonia floccosa, Rhizoctonia fragariae, Rhizoctonia fraxini, Rhizoctonia fusispora, Rhizoctonia globularis, Rhizoctonia gossypii, Rhizoctonia muneratii, Rhizoctonia papayae, Rhizoctonia quercus, Rhizoctonia repens, Rhizoctonia rubi, Rhizoctonia silvestris, Rhizoctonia solani, Phakopsora ampelopsidis, Phakopsora apoda, Phakopsora argentinensis, Phakopsora cherimoliae, Phakopsora cingens, Phakopsora coca, Phakopsora crotonis, Phakopsora euvitis, Phakopsora gossypii, Phakopsora hornotina, Phakopsora jatrophicola, Phakopsora meibomiae, Phakopsora meliosmae, Phakopsora meliosmae-myrianthae, Phakopsora montana, Phakopsora muscadiniae, Phakopsora myrtacearum, Phakopsora nishidana, Phakopsora orientalis, Phakopsora pachyrhizi, Phakopsora phyllanthi, Phakopsora tecta, Phakopsora uva, Phakopsora vitis, Phakopsora ziziphi-vulgaris,

Puccinia abrupta, Puccinia acetosae, Puccinia achnatheri-sibirici, Puccinia acroptili, Puccinia actaeae-agropyri, Puccinia actaeae-elymi, Puccinia antirrhini, Puccinia argentata, Puccinia arrhenatheri, Puccinia arrhenathericola, Puccinia artemisiae-keiskeanae, Puccinia arthrocnemi, Puccinia asteris, Puccinia atra, Puccinia aucta, Puccinia ballotiflora, Puccinia bartholomaei, Puccinia bistortae, Puccinia cacabata, Puccinia calcitrapae, Puccinia calthae, Puccinia calthicola, Puccinia calystegiae-soldanellae, Puccinia canaliculata, Puccinia caricis-montanae, Puccinia caricis-stipatae, Puccinia carthami, Puccinia cerinthes-agropyrina, Puccinia cesatii, Puccinia chrysanthemi, Puccinia circumdata, Puccinia clavata, Puccinia coleataeniae, Puccinia coronata, Puccinia coronati-agrostidis, Puccinia coronati-brevispora, Puccinia coronati-calamagrostidis, Puccinia coronati-hordei, Puccinia coronati-japonica, Puccinia coronati-longispora, Puccinia crotonopsidis, Puccinia cynodontis, Puccinia dactylidina, Puccinia dietelii, Puccinia digitata, Puccinia distincta, Puccinia duthiae, Puccinia emaculata, Puccinia erianthi, Puccinia eupatoriicolumbiani, Puccinia flavenscentis, Puccinia gastrolobii, Puccinia geitonoplesii, Puccinia gigantea, Puccinia glechomatis, Puccinia helianthi, Puccinia heterogenea, Puccinia heterospora, Puccinia hydrocotyles, Puccinia hysterium, Puccinia impatientis, Puccinia impedita, Puccinia imposita, Puccinia infra-aequatorialis, Puccinia insolita, Puccinia justiciae, Puccinia klugkistiana, Puccinia knersvlaktensis, Puccinia lantanae, Puccinia lateritia, Puccinia latimamma, Puccinia liberta, Puccinia littoralis, Puccinia lobata, Puccinia lophatheri, Puccinia loranthicola, Puccinia menthae, Puccinia mesembryanthemi, Puccinia meyeri-albertii, Puccinia miscanthi, Puccinia miscanthidii, Puccinia mixta, Puccinia montanensis, Puccinia morata, Puccinia morthieri, Puccinia nitida, Puccinia oenanthes-stoloniferae, Puccinia operta, Puccinia otzeniani, Puccinia patriniae, Puccinia pentstemonis, Puccinia persistens, Puccinia phyllostachydis, Puccinia pittieriana, Puccinia platyspora, Puccinia pritzeliana, Puccinia prostii, Puccinia pseudodigitata, Puccinia pseudostriiformis, Puccinia psychotriae, Puccinia punctata, Puccinia punctiformis, Puccinia recondita, Puccinia rhei-undulati, Puccinia rupestris, Puccinia senecionis-acutiformis, Puccinia septentrionalis, Puccinia setariae, Puccinia silvatica, Puccinia stipina, Puccinia stobaeae, Puccinia striiformis, Puccinia striiformoides, Puccinia stylidii, Puccinia substriata, Puccinia suzutake, Puccinia taeniatheri, Puccinia tageticola, Puccinia tanaceti, Puccinia tatarinovii, Puccinia tetragoniae, Puccinia thaliae, Puccinia thlaspeos, Puccinia tillandsiae, Puccinia tiritea, Puccinia tokyensis, Puccinia trebouxi, Puccinia triticina, Puccinia tubulosa, Puccinia tulipae, Puccinia tumidipes, Puccinia turgida, Puccinia urticae-acutae, Puccinia urticae-acutiformis, Puccinia urticae-caricis, Puccinia urticae-hirtae, Puccinia urticae-inflatae, Puccinia urticata, Puccinia vaginatae, Puccinia virgata, Puccinia xanthii, Puccinia xanthosiae, Puccinia zoysiae, more preferably of species Phakopsora pachyrhizi, Puccinia graminis, Puccinia striiformis, Puccinia hordei or Puccinia recondita,

more preferably of genus Phakopsora and most preferably Phakopsora pachyrhizi. As indicated above, fungi of these taxa are responsible for grave losses of crop yield. This applies in particular to rust fungi of genus Phakopsora. It is thus an advantage of the present invention that the method allows to reduce fungicide treatments against Phrakopsora pachyrhizi as described herein.

It is preferred according to the invention that the plant is a crop plant, preferably a dikotyledon, more preferably not of sub-family Solanoidae, more preferably not of family Solanaceae, more preferably a plant of order Fabales, more preferably a plant of family Fabaceae, more preferably a plant of tribus Phaseoleae, more preferably of genus Amphicarpaea, Cajanus, Canavalia, Dioclea, Erythrina, Glycine, Arachis, Lathyrus, Lens, Pisum, Vicia, Vigna, Phaseolus or Psophocarpus, even more preferably of species Amphicarpaea bracteata, Cajanus cajan, Canavalia brasiliensis, Canavalia ensiformis, Canavalia gladiata, Dioclea grandiflora, Erythrina latissima, Phaseolus acutifolius, Phaseolus lunatus, Phaseolus maculatus, Psophocarpus tetragonolobus, Vigna angularis, Vigna mungo, Vigna unguiculata, Glycine albicans, Glycine aphyonota, Glycine arenaria, Glycine argyrea, Glycine canescens, Glycine clandestina, Glycine curvata, Glycine cyrtoloba, Glycine dolichocarpa, Glycine falcata, Glycine gracei, Glycine hirticaulis, Glycine lactovirens, Glycine latifolia, Glycine latrobeana, Glycine microphylla, Glycine peratosa, Glycine pindanica, Glycine pullenii, Glycine rubiginosa, Glycine stenophita, Glycine syndetika, Glycine tabacina, Glycine tomentella, Glycine gracilis, Glycine max, Glycine max×Glycine soja, Glycine soja, more preferably of species Glycine gracilis, Glycine max, Glycine max×Glycine soja, Glycine soja, most preferably of species Glycine max. As shown herein particularly good yield improvements are obtained for soybean.

The crop may comprise, in addition to the heterologous expression cassette, one or more further heterologous elements. For example, transgenic soybean events comprising herbicide tolerance genes are for example, but not excluding others, GTS 40-3-2, MON87705, MON87708, MON87712, MON87769, MON89788, A2704-12, A2704-21, A5547-127, A5547-35, DP356043, DAS44406-6, DAS68416-4, DAS-81419-2, GU262, SYHTOH2, W62, W98, FG72 and CV127; transgenic soybean events comprising genes for insecticidal proteins are for example, but not excluding others, MON87701, MON87751 and DAS-81419. Cultivated plants comprising a modified oil content have been created by using the transgenes: gm-fad2-1, Pj.D6D, Nc. Fad3, fad2-1A and fatb1-A. Examples of soybean events comprising at least one of these genes are: 260-05, MON87705 and MON87769. Plants comprising such singular or stacked traits as well as the genes and events providing these traits are well known in the art. For example, detailed information as to the mutagenized or integrated genes and the respective events are available from websites of the organizations International Service for the Acquisition of Agrl. biotech Applications (ISAAA) (http://www.isaaa.org/gmapprovaldatabase) and the Center for Environmental Risk Assessment (CERA) (http://cera-qmc.org/GMCropDatabase). Further information on specific events and methods to detect them can be found for soybean events H7-1, MON89788, A2704-12, A5547-127, DP305423, DP356043, MON87701, MON87769, CV127, MON87705, DAS68416-4, MON87708, MON87712, SYHTOH2, DAS81419, DAS81419×DAS44406-6, MON87751 in WO04/074492, WO06/130436, WO06/108674, WO06/108675, WO08/054747, WO08/002872, WO09/064652, WO09/102873, WO10/080829, WO10/037016, WO11/066384, WO11/034704, WO12/051199, WO12/082548, WO13/016527, WO13/016516, WO14/201235.

The heterologous expression cassette according to the invention preferably comprises the respective Pti5 and/or SAR8.2 gene, or the Pti5-SAR8.2 fusion gene, operably linked to any of
a) a constitutively active promoter,
b) a tissue-specific or tissue-preferred promoter,
c) a promoter inducible by exposition of the plant to a pest, preferably a fungal pest.

A constitutively active promoter allows to provide the plant with expression of the Pti5 or SAR8.2 gene under mainly all circumstances and environmental conditions and in mainly all developmental stages of the plant (such as germling, mature plant or during flowering. Concerning tissue specific expression, a promoter can lead to a ubiquitous or tissue-specific expression of the Pti5 or SAR8.2 gene, respectively. Ubiquitous expression means that the gene of interest is expressed in mainly all tissues of the plant (such as root, stem, leaf or flower) A promoter with tissue specificity or preference provides such basal expression only or predominantly in the respective tissue. And an inducible promoter allows for a fast upregulation of expression upon exposition of the plant to the pest, thereby providing a fast reaction. Most preferably the plant in the method according to the present invention comprises the Pti5 and/or SAR8.2 gene in two copies, wherein one copy is under control of a constitutively active promoter, a tissue-specific or tissue-preferred promoter, and the other copy is under control of an inducible promoter, preferably a promoter inducible by exposition to the fungal pathogen, most preferably Phakopsora pachyrhizi. This way a comparatively low basal expression of the gene is ascertained, conserving metabolic resources, while defenses against significant pest exposure are ramped up when needed, thereby consuming metabolic resources for gene expression mainly when there is a significant exposure to the stress.

The invention also provides a method for producing a hybrid plant having improved yield relative to a control plant, comprising
i) providing
i-a) a first plant material comprising a Pti5 and a SAR8.2 gene, preferably comprising a heterologous Pti5 expression cassette and a heterologous SAR8.2 expression cassette, and a second plant material not comprising both a Pti5 and a SAR8.2 gene, or
i-b) a first plant material comprising a Pti5 gene, preferably comprising a heterologous Pti5 expression cassette, and a second plant material comprising a SAR8.2 gene, preferably comprising a heterologous SAR8.2 expression cassette, ii) producing an F1 generation from a cross of the first and second plant material, and
iii) selecting one or more members of the F1 generation that comprises said heterologous expression cassette.

As described herein, such hybrids allow to materialize the advantages conveyed by plants of the present invention, in particular the increase in yield, preferably seed mass yield, in normal field growth conditions, more preferably under at least low pathogen pressure, more preferably under at least low fungal pathogen pressure during the growth season.

It is a particular advantage of the present invention that the methods of the present invention do not require homozygous plants expressing the Pti5 and SAR8.2 genes but is also applicable for hemizygous or heterozygous plants. Correspondingly the hybrid production method of the present invention advantageously provides hybrid plants comprising both the advantageous heterologous expression cassette of the present invention and advantageous traits of the second plant material. Thus the hybrid production method according to the present invention allows to construct, with low effort, hybrids adapted to expected growth conditions for the next growth season.

The invention is hereinafter further described by way of examples and selected preferred embodiments. Neither the examples nor the selected embodiments are intended to limit the scope of the claims.

### EXAMPLES

### Example 1: Obtaining of Transformed Soybean Plants

All steps leading to the generation and first evaluation of the transformed soybean plants expressing single gene constructs described in this document, such as:
Isolation or synthesis of the respective genes
Generation of vectors for plant transformation
Transformation of the respective vectors in soybean plants
Evaluation of resistance of the transformed plants against soybean rust fungus are described in
   WO2014118018 (resistance gene: EIN2), examples 2, 3 and 6
   WO2013001435 (resistance gene: Pti5), examples 2, 3 and 6 (here: SEQ ID NO. 3)
   WO2014076614 (resistance gene: CaSAR=SAR8.2), examples 2, 3 and 6 (here: SEQ ID NO. 5)
   WO2014024079 (resistance gene: RLK2), examples 2, 3 and 6.
   WO2012023099 (resistance gene: ADR1)

Cloning of double gene stack constructs
a) SAR8.2 and Pti5
b) SAR8.2 and RLK2
c) Pti5 and ADR1
d) Pti5 and EIN2
e) Pti5 and RLK2

The single gene cassettes (promoter gene terminator) were cloned as described in the patents shown above. As all components and the entire cassette are flanked by unique eight-base restriction enzymes, we cut out the entire expression cassette and transferred it into a three-way GATEWAY compatible p-Entry vector ((Gateway system, Invitrogen, Life Technologies, Carlsbad, California, USA).

All double gene constructs were generated by using a three-way gateway reaction. To generate the binary plant transformation vector containing both single gene cassettes, a triple LR reaction (Gateway system, Invitrogen, Life Technologies, Carlsbad, California, USA) was performed according to manufacturer's protocol by using:
a) the first single gene cassette located in a pENTRY vector between ATT4 and ATT1 recombination sites,
b) an empty pENTRY vector, having ATT1 and ATT2 recombination sites,
c) the second single gene cassette located in a pENTRY vector between ATT2 and ATT3 recombination sites and
d) a target a binary pDEST vector containing ATT4 and ATT3 recombination sites. In addition the pDEST vector contained: (1) a spectinomycin/streptomycin resistance cassette for bacterial selection (2) a pVS1 origin for replication in Agrobacteria (3) a ColE1 origin of replication for stable maintenance in E. coli and (4) between the right and left border an AHAS selection under control of an AtAHASL-promoter.

The recombination reaction was transformed into E. coli (DH5alpha), mini-prepped and screened by specific restriction digestions. A positive clone from each vector construct was sequenced and submitted soybean transformation. The soybean transformation was performed as described in the single gene patents above.

Where the above documents refer to more than one transformation method in example 3, the result in terms of yield and resistance to Phakopsora pachyrhizi were found independently of the transformation method employed.

Based on the result of the evaluation of resistance against soybean rust in TO and/or T1 generation, the most resistance and phenotypically best looking 3-5 events were selected for further analysis.

Homozygous T2 or T3 seeds were used for field trials. To obtain homozygous seeds, segregating T1 seeds of the selected 3-5 events per construct were planted. Individual plants that were homozygous for the transgene were selected by using TaqMan® PCR assay as described by the manufacturer of the assay (Thermo Fisher Scientific, Waltham, MA USA 02451).

10-30 homozygous plants per event were grown under standard conditions (12 h daylength, 25° C.) and selfed (in-bred). Mature homozygous seeds were harvested approx. 120 days after planting. Harvested seeds of all 10-30 homozygous plants per event were pooled.

### Example 2: Field Trials

Homozygous T3 seeds of 3-5 events per construct were tested in the field for resistance against soybean rust, yield and agronomic performance.

Field trials were performed in Brazil on two sites in the states of Sao Paulo and Minas Gerais, respectively. Field trials were planted depending on weather conditions in November or early December (Safra season) or early February (Safrinha season) to ensure sufficient inoculum of Asian soybean rust.

Material was tested in split plot trials (2 m long, 4 rows per plot), 3-4 replications per event and trial site. Field trials to test trait performance were grown using standard cultural practice, e.g. in terms of weed and insect control and fertilization.

Depending on the trial, 2 different fungicide related treatment were made:
1. No fungicide treatment (“untreated”)
2. One fungicide application (“A Treatment”) at the onset of ASR diseases (˜35-45 days after planting, depending on location, planting date and year). The fungicide treatment reduced ASR disease severity in the early season allowing to test the trait efficacy under different ASR pressure at the same location, simulating a year with less disease or later disease onset.

About 10% of the plots were used as control. Depending on trial design, the untransformed wildtype (WT) mother line or bulk of seeds harvested from null-segregants, grown in parallel to the transgenic mother plants (see above) were used as control.

### Example 3: ASR Rating

Asian soybean rust (ASR) infection was rated by experts using the scheme published by Godoy et al (2006) (citation Godoy, C., Koga, L., Canteri, M. (2006) Diagrammatic scale for assessment of soybean rust severity, Fitopatologia Brasileira 31(1)).

To eliminate transgene insertion effects, which would be only dependent on the integration locus, 3 to 5 independent transgenic events were assessed per field trial (Event=offspring of a single plant having taken up the heterologous expression cassettes from the same vector construct but integrated at different genomic loci).

The three canopy levels (lower, middle and upper canopy) were rated independently and the average of the infection of all three canopy levels is counted as infection. In total, 4-7 ratings over the course of a whole growth season were performed, starting at the early onset of disease and repeated every 6-8 days; if weather was not suitable for disease progression the time in between 2 ratings was elongated to at most 22 days.

To eliminate transgene insertion effects, which would be only dependent on the integration locus, 3 to 5 independent transgenic events were assessed per field trial.

To compare the disease progression in different events over the season we calculated the Area Under Disease Progression Curve (AUDPC) on the basis of the infection ratings (for reference see: M. J. Jeger and S. L. H. Viljanen-Rollinson (2001) The use of the area under the diseaseprogress curve (AUDPC) to assess quantitative disease resistance in crop cultivars Theor Appl Genet 102:32-40.)

The AUDPC is a quantitative value describing the disease intensity over the complete season. To calculate the AUDPC a series of disease ratings are taken over the season. The AUDPC represents the sum of all averages of 2 consecutive ratings that are multiplied by the time between the ratings

$$AUDPC = \qquad {{\sum }_{i = 1}}^{n - 1} \qquad ( \frac{{y}_{i} + {y}_{i + 1}}{2} ) \qquad ( {t}_{i + 1} - {t}_{i} )$$

“t”: the time of each disease rating in days after planting
“y”: percentage of diseased leaf area over all canopy levels
“n”: number of disease ratings

To calculate the relative disease resistance the following formula was used:

Relative disease resistance=(AUDPC(control)/AUDPC(event))−1)*100%

The relative disease resistance on gene (construct) level was calculated by averaging the relative disease resistance (based on formula above) of the 3-5 events expressing the same gene (=same construct).

TABLE 1
Average resistance of soybean plants against Phakopsora
pachyrhizi in 2 independent field trials

| | | Location 1 | | Location 2 | |
|---|---|---|---|---|---|
| | | | relative | | relative |
| | | | resistance | | resistance |
| Treatment | Genes | AUDPC | [%] | AUDPC | [%] |
| Treatment A | Pti5 | 1815.7 | 25.6% | 1689 | 3.7% |
| Treatment A | SAR8.2 | 2282.1 | 6.4% | 1716 | 2.2% |
| Treatment A | Pti5::SAR8.2 | 1910.4 | 21.7% | 1590 | 9.4% |
| Treatment A | WT | 2439.1 | 0.0% | 1754 | 0.0% |
| Untreated | Pti5 | 2417.4 | 24.6% | 2022 | 5.4% |
| Untreated | SAR8.2 | 3178.5 | 0.9% | 2029 | 5.1% |
| Untreated | Pti5::SAR8.2 | 2729.2 | 14.9% | 1847 | 13.6% |
| Untreated | WT | 3207.6 | 0.0% | 2138 | 0.0% |

Both single Pti5 and SAR8.2 genes and the molecular stack of Pti5+SAR8.2 increased the resistance in both locations and under both treatments (untreated and one fungicide treatment (“Treatment A”), but the combination of both genes clearly did not lead to an additive or more than additive increase of resistance

### Example 4: Determination of Yield

For yield determination only the 2 middle rows per plot (see above) were harvested to reduce overestimation by edge effects. A combine was used which was able to record the total grain weight of the plot and grain moisture. After moisture correction the grain yield was calculated from kg/plot to kg/ha.

Overexpression of SAR8.2, Pti5 and RLK2 as single gene constructs significantly increased yield of soybean when infected by soybean rust. In addition, we could show that there is some yield increase mediated by the overexpression of the ADR1 gene (see FIG. 4). To determine the effect of combining efficacious lead genes in a molecular stack, a comparative trial was performed in a way to allow a head-to-head comparison of plants expressing lead gene stacks to plants expressing the respective single genes.

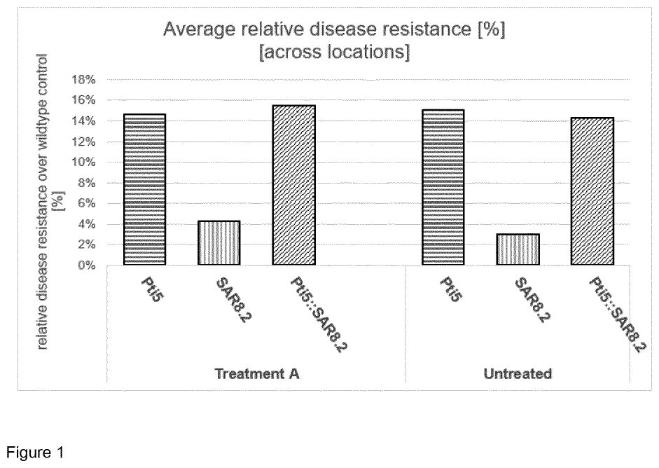

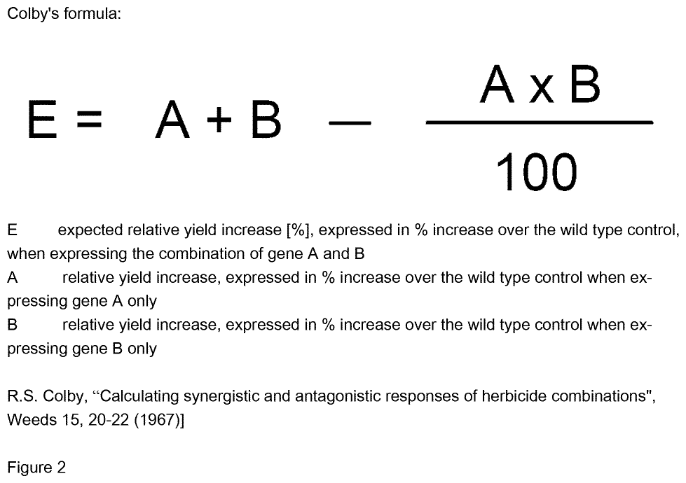

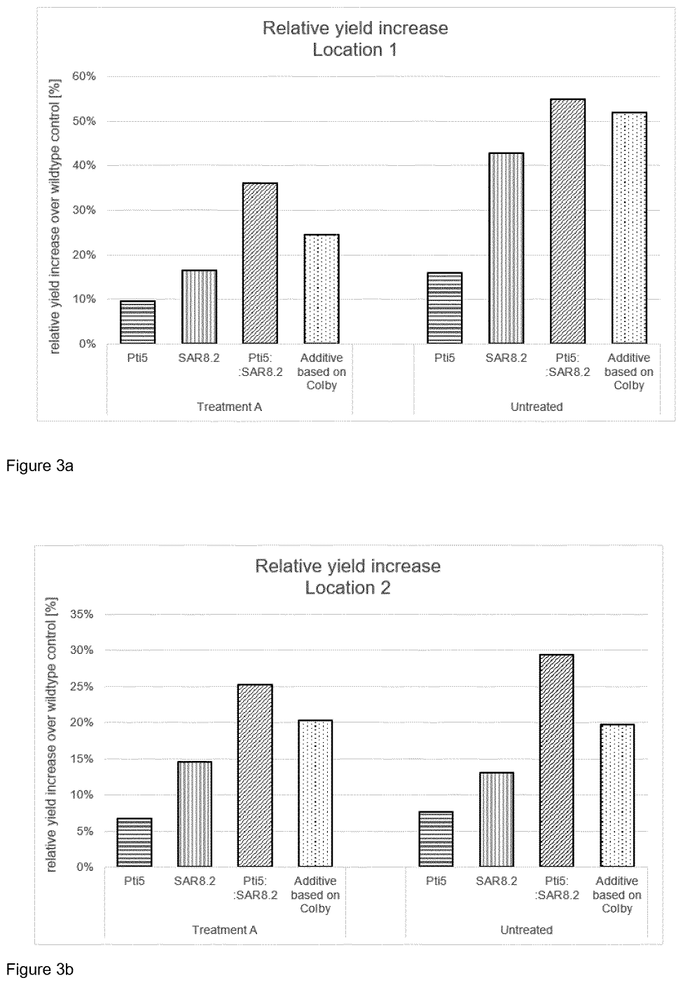

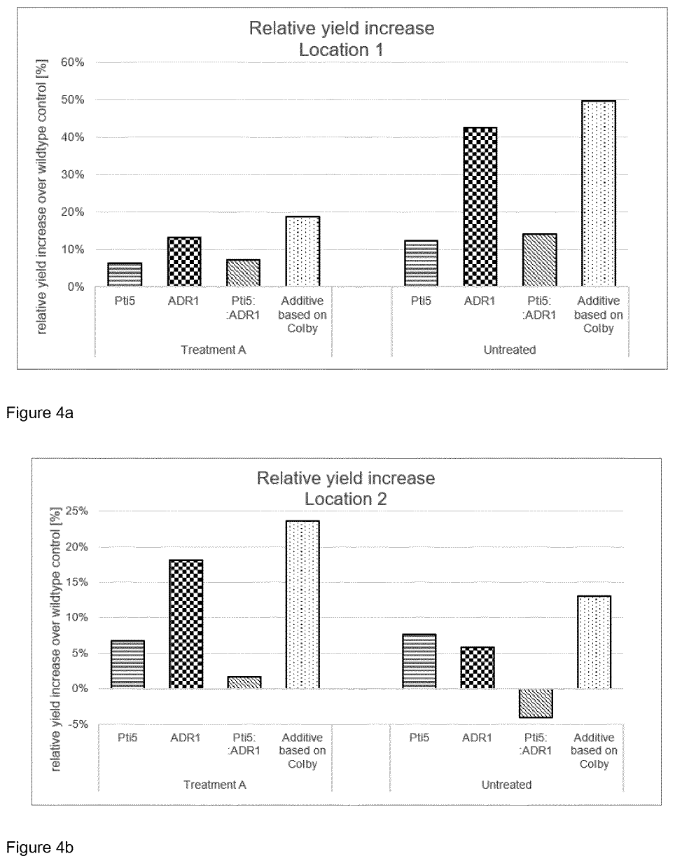

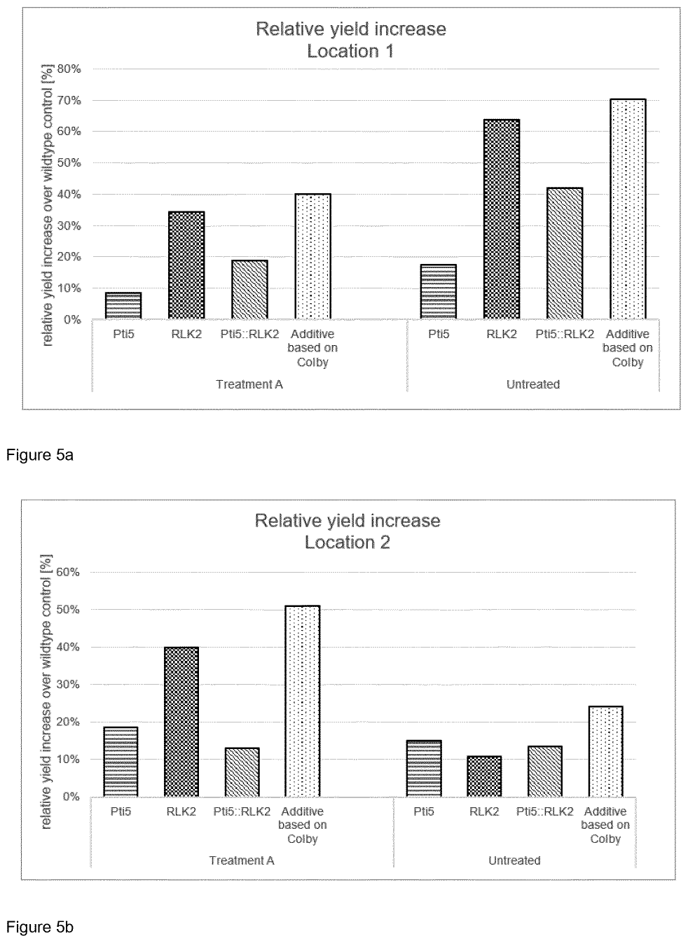

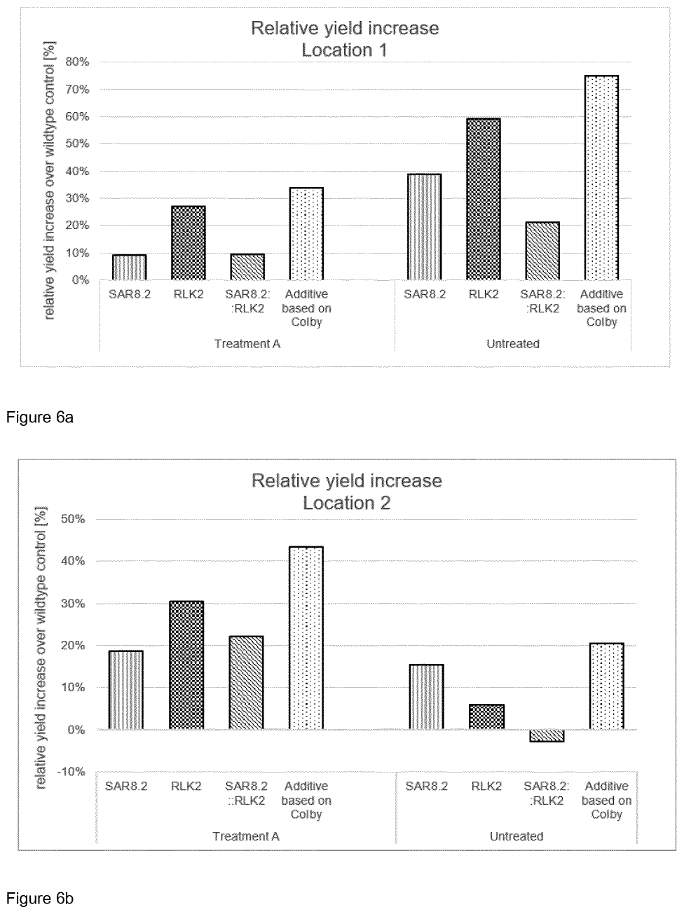

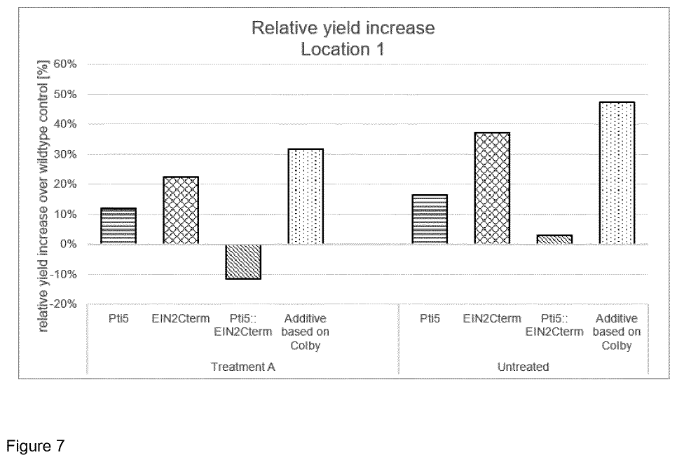

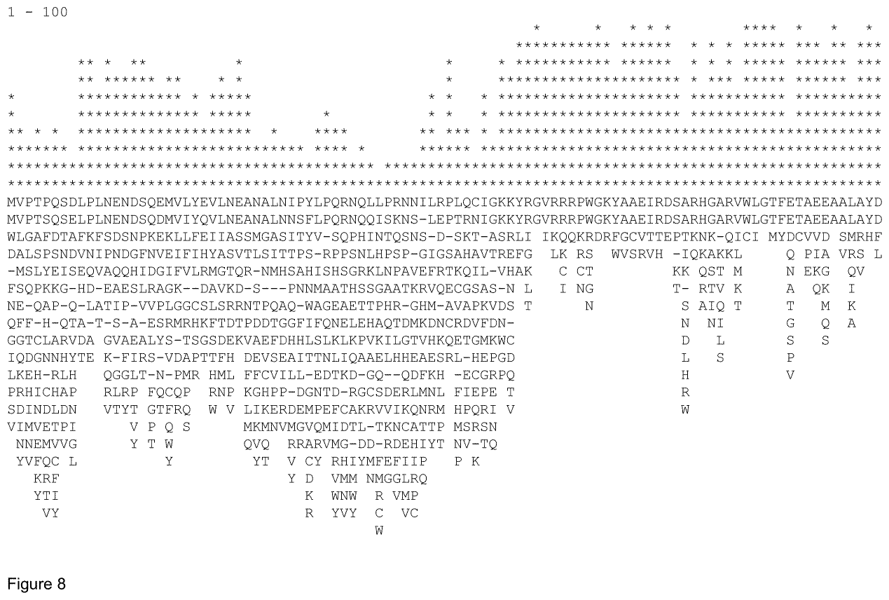

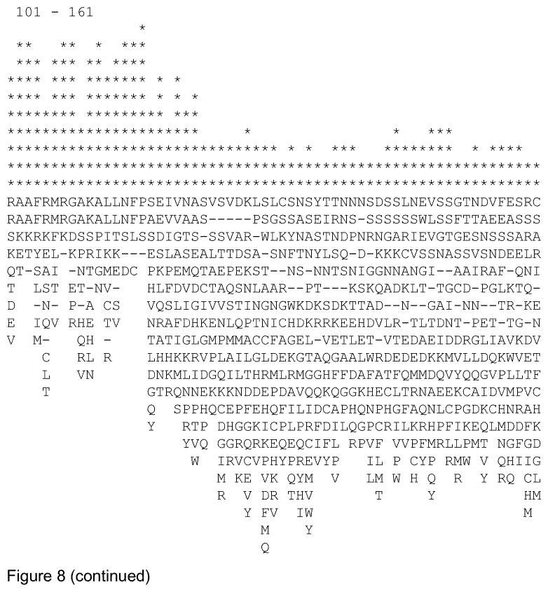

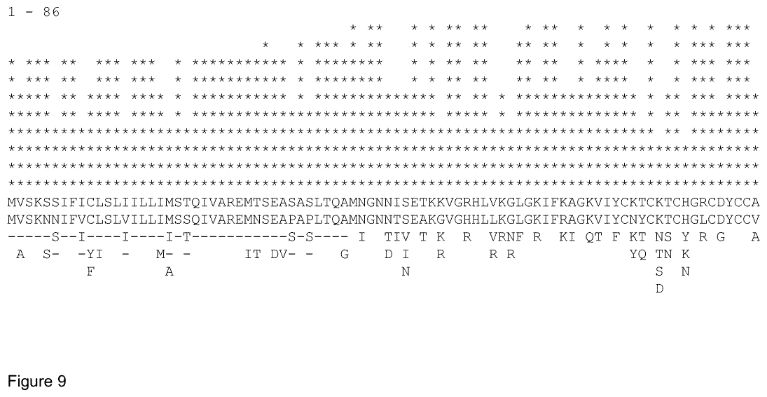

Surprisingly the combination of Pti5 and SAR8.2 led to a strong increase in yield of in average 36% (average of both treatments and both locations, for specific values see table below). This strong increase was not expected based on the disease resistance data (see example 3 and FIG. 1) or based on the yield increase of both single gene controls SAR8.2.

TABLE 2
Yield increases of soybeans harvested in 2 independent field trials

| | | Location 1 | | Location 2 | |
|---|---|---|---|---|---|
| | | | relative | | relative |
| | | Yield | yield | Yield | yield |
| Treatment | Genes | (kg/ha) | increase | (kg/ha) | increase |
| A | Pti5 | 2140 | 10% | 2409 | 7% |
| A | SAR8.2 | 2276 | 17% | 2586 | 15% |
| A | Pti5::SAR8.2 | 2658 | 36% | 2828 | 25% |
| A | WT | 1953 | 0% | 2257 | 0% |
| Untreated | Pti5 | 1420 | 16% | 1713 | 8% |
| Untreated | SAR8.2 | 1748 | 43% | 1799 | 13% |
| Untreated | Pti5::SAR8.2 | 1896 | 55% | 2059 | 29% |
| Untreated | WT | 1224 | 0% | 1591 | 0% |

The measured yields of each variant (construct×treatment×location) were compared to the yield of the non-transgenic wild type control (WT), to calculate the relative yield increases per construct, treatment and location by using the formula:

Relative yield increase [%]=(yield of variant [kg]/yield of respective WT [kg]−1)*100%

The expected yield increase of the combination of the 2 genes was determined using Colby's formula [R. S. Colby, “Calculating synergistic and antagonistic responses of herbicide combinations”, Weeds 15, 20-22 (1967)] and compared with the observed yield increases. Colby's formula predicts a value of a combination based on the result of both single factors (here: genes) that represents a fully additive interaction of both factors. Values greater than this value can be considered as resulting from a more than additive interaction.

Colby's formula:

$$E = \qquad A + B \qquad - \qquad \frac{A \times B}{100}$$

E expected relative yield increase, expressed in % increase over the wild type control, when expressing the combination of gene A and B
A relative yield increase, expressed in % increase over the wild type control when expressing gene A only
B relative yield increase, expressed in % increase over the wild type control when expressing gene B only

Using the Colby formula and calculating the additive values for the combination of SAR8.2 and Pti5 results in:
a) Location 1, Treatment A:
   yield increase predicted by Colby formula: 24.6%%
   measured yield increase: 36.1%
b) Location 1, untreated:
   yield increase predicted by Colby formula: 51.9% measured
   yield increase: 54.8%
c) Location 2, Treatment A:
   yield increase predicted by Colby formula: 20.3%%
   measured yield increase: 25.3%
d) Location 2, untreated:
   yield increase predicted by Colby formula: 19.8% measured
   yield increase: 29.4%

As clearly visible above and in FIG. 3a, 3b the combination of SAR8.2 and Pti5 leads, in all locations and treatments, to a yield increase that is larger than predicted by the Colby formula and therefore more than additive.

This result was highly surprising and not foreseeable, because the mode of action of Pti5 and SAR8.2 are completely different. The SAR8.2 protein is described to act as an antifungal protein, whereas Pti5 is a transcription factor working in the regulation of defense reactions.

None of the other lead gene combinations tested in parallel showed a comparable result, indicating that the yield increase caused by the combination of SAR8.2 and Pti5 expression is an extraordinary advantage of the present invention.

Analyzing further results of the comparative field trial, it turned out that even though most of the selected individual resistance genes led to an increased yield in nearly all locations or treatments in comparison to the non transgenic wild type (see table 3-6, and FIG. 4-7), no combination (stack) of resistance genes showed a yield increase that can be considered additive (or more than additive) based on the result of the single gene constructs.

TABLE 3
Yields of soybean plants comprising
a combination of SAR8.2 and RLK2

| Treatment | Gene | Location 1 | Location 2 |
|---|---|---|---|
| Treatment A | SAR8.2 | 9% | 19% |
| Treatment A | RLK2 | 27% | 30% |
| Treatment A | SAR8.2::RLK2 | 10% | 22% |
| | Additive based on Colby | 34% | 43% |
| Untreated | SAR8.2 | 39% | 15% |
| Untreated | RLK2 | 59% | 6% |
| Untreated | SAR8.2::RLK2 | 21% | −3% |
| | Additive based on Colby | 75% | 21% |

TABLE 4
Yields of soybean plants comprising
a combination of Pti5 and ADR1

| Treatment | Gene | Location 1 | Location 2 |
|---|---|---|---|
| Treatment A | Pti5 | 6% | 7% |
| Treatment A | ADR1 | 13% | 18% |
| Treatment A | Pti5::ADR1 | 7% | 2% |
| | Additive based on Colby | 19% | 24% |
| Untreated | Pti5 | 12% | 8% |
| Untreated | ADR1 | 43% | 6% |
| Untreated | Pti5::ADR1 | 14% | −4% |
| | Additive based on Colby | 50% | 13% |

TABLE 5
Yields of soybean plants comprising a combination
of Pti5 and EIN2(=”AtEIN2Cterm”)

| Treatment | Gene | Location 1 | Location 2 |
|---|---|---|---|
| Treatment A | Pti5 | 12% | — |
| Treatment A | AtEIN2Cterm | 22% | — |
| Treatment A | Pti5::AtEIN2Cterm | −12% | — |
| | Additive based on Colby | 32% | — |
| Untreated | Pti5 | 16% | — |
| Untreated | AtEIN2Cterm | 37% | — |
| Untreated | Pti5::AtEIN2Cterm | 3% | — |
| | Additive based on Colby | 47% | — |

TABLE 6
Yields of soybean plants comprising
a combination of Pti5 and RLK2

| Treatment | Gene | Location 1 | Location 2 |
|---|---|---|---|
| Treatment A | Pti5 | 9% | 19% |
| Treatment A | RLK2 | 34% | 40% |
| Treatment A | Pti5::RLK2 | 19% | 13% |
| | Additive based on Colby | 40% | 51% |
| Untreated | Pti5 | 17% | 15% |
| Untreated | RLK2 | 64% | 11% |
| Untreated | Pti5::RLK2 | 42% | 14% |
| | Additive based on Colby | 70% | 24% |

1. Method for improving the yield produced by a plant relative to a control plant, comprising
i) providing a plant comprising a Pti5 and a SAR8.2 gene and/or a Pti5-SAR8.2 fusion gene, wherein preferably the Pti5 and/or SAR8.2 genes are provided in a respective heterologous expression cassette, and
ii) cultivating the plant.

2. Farming method for improving the yield produced by a plant relative to a control plant, comprising cultivation of a plant comprising a Pti5 and SAR8.2 gene and/or a Pti5-SAR8.2 fusion gene, wherein the plant (a) overexpresses Pti5 and SAR8.2 and/or (b) comprises a heterologous Pti5 expression cassette and/or a heterologous SAR8.2 expression cassette and/or (c) expresses a heterologous Pti5 and/or heterologous SAR8.2 gene, wherein during cultivation of the plant the number of pesticide treatments per growth season is reduced by at least one relative to the control plant, preferably by at least two.

3. Method according to claim 1, wherein
the yield is one or more of
biomass per area,
grain mass per area,
seed mass per area.

4. Method according to claim 1, wherein the yield is increased in the presence of a pest relative to a control plant.

5. Method according to claim 1, wherein the pest is or comprises at least a fungal pest.

6. Method according to claim 1, wherein the plant is a crop plant.

7. Method according to claim 1, wherein the plant comprises a heterologous Pti5 expression cassette and/or a heterologous SAR8.2 expression cassette, wherein for each expression cassette the respective Pti5 or SAR8.2 gene is operably linked to any of
a) a constitutively active promoter,
b) a tissue-specific or tissue-preferred promoter,
c) a promoter inducible by exposition of the plant to a pest.

8. Method according to claim 1, wherein the cultivation is performed on an ensemble of at least 1000 plants.

9. Plant cell, plant part or whole plant comprising a Pti5 and a SAR8.2 gene and/or a Pti5-SAR8.2 fusion gene.

10. (canceled)

11. (canceled)

12. Method of synergistic yield improvement comprising providing, in a plant cell, plant part or plant at least a Pti5 protein and a SAR8.2 protein.
